(19) United States
(12) Patent Application Publication
(10) Pub. No.: US 2012/0251518 A1
(43) Pub. Date: Oct. 4, 2012

(54) Endopeptidase Treatment of Sexual Dysfunction Disorders

(75) Inventors: Andrew M. Blumenfeld, Del Mar, CA (US); Mitchell F. Brin, Newport Beach, CA (US)

(73) Assignee: ALLERGAN, INC., Irvine, CA (US)

(21) Appl. No.: 13/425,281

(22) Filed: Mar. 20, 2012

Related U.S. Application Data
(60) Provisional application No. 61/469,011, filed on Mar. 29, 2011.

(51) Int. Cl. A61K 38/48; A61P 15/00; A61P 15/12; C12N 9/48

(52) U.S. Cl. 424/94.63; 435/212

(57) ABSTRACT

The present specification discloses TEMs, compositions comprising such TEMs, compositions comprising such TEMs and Clostridial toxins, methods of treating a sexual dysfunction disorder in an individual using such compositions, use of such TEMs in manufacturing a medicament for treating a sexual dysfunction disorder, use of such TEMs and Clostridial toxins in manufacturing a medicament for treating a sexual dysfunction disorder, use of such TEMs in treating a sexual dysfunction disorder, and use of such TEMs and Clostridial toxins in treating a sexual dysfunction disorder.

This application claims the benefit of priority pursuant to 35 U.S.C. §119(e) to U.S. provisional patent application Ser. No. 61/469,011, filed Mar. 29, 2011, incorporated entirely by reference.

Sexual activity for most people is a positive experience and an important aspect of their sexuality. Generally, a person can normally control how they will respond to sexual stimuli. They will normally know what activities or situations are erotic, and can if they so choose can either create or avoid these stimuli. Similarly, a person's sexual partner will also typically know his or her partners erotic stimuli and turn-offs. Normally, a person does not experience desire, arousal, and/or orgasm on every occasion that there is exposure to sexual stimuli, nor respond in a sexual way every time such exposure occurs. These situations are considered normal, but depend on the maturity, age, culture and other factors influencing the person.

However, when a person fails to be sexually stimulated in a situation that would normally produce a sexual response, and this lack of response is persistent, it may be due to a sexual dysfunction disorder. There are many reasons why a person fails to be sexually stimulated, including, e.g., a mental disorder, substance misuse, a medical condition or physical condition. The lack of sexual stimulation may be due to a lack of sexual desire, sexual arousal, and/or the inability to have an orgasm. A person may always have had no or low response to a sexual stimulus or the lack of a sexual response may have been acquired during the person's life.

Similarly, a sexual dysfunction disorder may be the underlying cause of a person who becomes sexually stimulated in a situation that would normally not produce a sexual response in a typical person. For example, a person may be hypersexual, which is a desire to engage in sexual activities considered abnormally high in relation to normal development or culture, or suffering from a persistent genital arousal disorder, which is a spontaneous, persistent, and uncontrollable arousal, and the physiological changes associated with arousal. Another problem which some people have in controlling their level of arousal is referred to as sexual addiction.

The ability of Clostridial toxins, such as, e.g., Botulinum neurotoxins (BoNTs), BoNT/A, BoNT/B, BoNT/C1, BoNT/D, BoNT/E, BoNT/F and BoNT/G, and Tetanus neurotoxin (TeNT), to inhibit neuronal transmission are being exploited in a wide variety of therapeutic and cosmetic applications, see e.g., William J. Lipham, COSMETIC AND CLINICAL APPLICATIONS OF BOTULINUM TOXIN (Slack, Inc., 2004). Clostridial toxins commercially available as pharmaceutical compositions include, BoNT/A preparations, such as, e.g., BOTOX® (Allergan, Inc., Irvine, Calif.), DYSPORT®/RELOXIN®, (Beaufour Ipsen, Porton Down, England), NEURONOX® (Medy-Tox, Inc., Ochang-myeon, South Korea), BTX-A (Lanzhou Institute Biological Products, China) and XEOMIN® (Merz Pharmaceuticals, GmbH., Frankfurt, Germany); and BoNT/B preparations, such as, e.g., MYOBLOC™/NEUROBLOC™ (Solstice Neurosciences, Inc., South San Francisco, Calif.). As an example, BOTOX® is currently approved in one or more countries for the following indications: achalasia, adult spasticity, anal fissure, back pain, blepharospasm, bruxism, cervical dystonia, essential tremor, glabellar lines or hyperkinetic facial lines, headache, hemifacial spasm, hyperactivity of bladder, hyperhidrosis, juvenile cerebral palsy, multiple sclerosis, myoclonic disorders, nasal labial lines, spasmodic dysphonia, strabismus and VII nerve disorder.

Clostridial toxin therapies are successfully used for many indications. Generally, administration of a Clostridial toxin treatment is well tolerated. However, toxin administration in some applications can be challenging because of the larger doses required to achieve a beneficial effect. Larger doses can increase the likelihood that the toxin may move through the interstitial fluids and the circulatory systems, such as, e.g., the cardiovascular system and the lymphatic system, of the body, resulting in the undesirable dispersal of the toxin to areas not targeted for toxin treatment. Such dispersal can lead to undesirable side effects, such as, e.g., inhibition of neurotransmitter release in neurons not targeted for treatment or paralysis of a muscle not targeted for treatment. For example, a individual administered a therapeutically effective amount of a BoNT/A treatment into the neck muscles for cervical dystonia may develop dysphagia because of dispersal of the toxin into the oropharynx. As another example, a individual administered a therapeutically effective amount of a BoNT/A treatment into the bladder for overactive bladder may develop dry mouth and/or dry eyes. Thus, there still remains a need for treatments having the therapeutic effects that only larger doses of a Clostridial toxin can currently provide, but reduce or prevent the undesirable side-effects associated with larger doses of a Clostridial toxin administration.

A Clostridial toxin treatment inhibits neurotransmitter release by disrupting the exocytotic process used to secret the neurotransmitter into the synaptic cleft. There is a great desire by the pharmaceutical industry to expand the use of Clostridial toxin therapies beyond its current myo-relaxant applications to treat sensory, sympathetic, and/or parasympathetic nerve-based ailments, such as, e.g., various kinds of sexual dysfunction disorders. One approach that is currently being exploited involves modifying a Clostridial toxin such that the modified toxin has an altered cell targeting capability for a neuronal or non-neuronal cell of interest. Called re-targeted endopeptidases or Targeted Vesicular Exocytosis Modulator Proteins (TVEMPs) or Targeted Exocytosis Modulators (TEMs), these molecules achieve their exocytosis inhibitory effects by targeting a receptor present on the neuronal or non-neuronal target cell of interest. This re-targeted capability is achieved by replacing the naturally-occurring binding domain of a Clostridial toxin with a targeting domain showing a selective binding activity for a non-Clostridial toxin receptor present in a cell of interest. Such modifications to the binding domain result in a molecule that is able to selectively bind to a non-Clostridial toxin receptor present on the target cell. A re-targeted endopeptidase can bind to a target receptor, translocate into the cytoplasm, and exert its proteolytic effect on the SNARE complex of the neuronal or non-neuronal target cell of interest.

The present specification discloses TEMs, compositions comprising TEMs, and methods for treating an individual suffering from a sexual dysfunction disorder. This is accomplished by administering a therapeutically effective amount of a composition comprising a TEM to an individual in need thereof. The disclosed methods provide a safe, inexpensive, out patient-based treatment for the treatment of sexual dysfunction disorders. In addition, the therapies disclosed herein reduce or prevent unwanted side-effects associated with larger Clostridial toxin doses. These and related advantages are useful for various clinical applications, such as, e.g., the treatment of sexual dysfunction disorders where a larger amount of a Clostridial toxin to an individual could produce a beneficial effect, but for the undesirable side-effects.

### SUMMARY

With reference to sexual dysfunction disorders as disclosed herein, and without wishing to be limited by any particular theory, it is believed that sympathetic, parasympathetic, and/or sensory neurons have important functions in aspects of sensory perception and that improper innervations from these types of neurons can contribute to one or more different types of sexual dysfunction disorders. As such, TEMs comprising a targeting domain for a receptor present on sympathetic, parasympathetic, and/or sensory neurons can reduce or prevent these improper innervations, thereby reducing or preventing one or more symptoms associate with a sexual dysfunction disorder.

Aspects of the present specification disclose methods of treating a sexual dysfunction disorder in an individual, the methods comprising the step of administering to the individual in need thereof a therapeutically effective amount of a composition including a TEM, wherein administration of the composition reduces a symptom of the sexual dysfunction disorder, thereby treating the individual. In some aspects, a TEM may comprise a targeting domain, a Clostridial toxin translocation domain and a Clostridial toxin enzymatic domain. In some aspects, a TEM may comprise a targeting domain, a Clostridial toxin translocation domain, a Clostridial toxin enzymatic domain, and an exogenous protease cleavage site. A targeting domain includes, without limitation, a sensory neuron targeting domain, a sympathetic neuron targeting domain, or a parasympathetic neuron targeting domain. A sexual dysfunction disorder includes, without limitation, a sexual desire disorder, a sexual arousal disorder, a sexual orgasm disorder, a sexual pain disorder, a sexsomnia, or a climacturia.

Other aspects of the present specification disclose uses of a TEM disclosed herein in the manufacturing a medicament for treating a sexual dysfunction disorder disclosed herein in an individual in need thereof.

Yet other aspects of the present specification uses of a TEM disclosed herein in the treatment of a sexual dysfunction disorder disclosed herein in an individual in need thereof.

Other aspects of the present specification disclose methods of treating a sexual dysfunction disorder in an individual, the methods comprising the step of administering to the individual in need thereof a therapeutically effective amount of a composition including a Clostridial neurotoxin and a TEM, wherein administration of the composition reduces a symptom of the sexual dysfunction, thereby treating the individual. A Clostridial neurotoxin includes, without limitation, a Botulinum toxin (BoNT), a Tetanus toxin (TeNT), a Baratii toxin (BaNT), and a Butyricum toxin (BuNT). In some aspects, a TEM may comprise a targeting domain, a Clostridial toxin translocation domain and a Clostridial toxin enzymatic domain. In some aspects, a TEM may comprise a targeting domain, a Clostridial toxin translocation domain, a Clostridial toxin enzymatic domain, and an exogenous protease cleavage site. A targeting domain includes, without limitation, a sensory neuron targeting domain, a sympathetic neuron targeting domain, or a parasympathetic neuron targeting domain. A sexual dysfunction disorder includes, without limitation, a sexual desire disorder, a sexual arousal disorder, a sexual orgasm disorder, a sexual pain disorder, a sexsomnia, or a climacturia.

Other aspects of the present specification disclose uses of a Clostridial neurotoxin and a TEM disclosed herein in the manufacturing a medicament for treating a sexual dysfunction disorder disclosed herein in an individual in need thereof.

Yet other aspects of the present specification uses of a Clostridial neurotoxin and a TEM disclosed herein in the treatment of a sexual dysfunction disorder disclosed herein in an individual in need thereof.

### BRIEF DESCRIPTION OF THE DRAWINGS

FIG. 1 shows a schematic of the current paradigm of neurotransmitter release and Clostridial toxin intoxication in a central and peripheral neuron. FIG. 1A shows a schematic for the neurotransmitter release mechanism of a central and peripheral neuron. The release process can be described as comprising two steps: 1) vesicle docking, where the vesicle-bound SNARE protein of a vesicle containing neurotransmitter molecules associates with the membrane-bound SNARE proteins located at the plasma membrane; and 2) neurotransmitter release, where the vesicle fuses with the plasma membrane and the neurotransmitter molecules are exocytosed. FIG. 1B shows a schematic of the intoxication mechanism for tetanus and botulinum toxin activity in a central and peripheral neuron. This intoxication process can be described as comprising four steps: 1) receptor binding, where a Clostridial toxin binds to a Clostridial receptor system and initiates the intoxication process; 2) complex internalization, where after toxin binding, a vesicle containing the toxin/receptor system complex is endocytosed into the cell; 3) light chain translocation, where multiple events are thought to occur, including, e.g., changes in the internal pH of the vesicle, formation of a channel pore comprising the HN domain of the Clostridial toxin heavy chain, separation of the Clostridial toxin light chain from the heavy chain, and release of the active light chain and 4) enzymatic target modification, where the activate light chain of Clostridial toxin proteolytically cleaves its target SNARE substrate, such as, e.g., SNAP-25, VAMP or Syntaxin, thereby preventing vesicle docking and neurotransmitter release.

FIG. 2 shows the domain organization of naturally-occurring Clostridial toxins. The single-chain form depicts the amino to carboxyl linear organization comprising an enzymatic domain, a translocation domain, and a retargeted peptide binding domain. The di-chain loop region located between the translocation and enzymatic domains is depicted by the double SS bracket. This region comprises an endogenous di-chain loop protease cleavage site that upon proteolytic cleavage with a naturally-occurring protease, such as, e.g., an endogenous Clostridial toxin protease or a naturally-occurring protease produced in the environment, converts the single-chain form of the toxin into the di-chain form. Above the single-chain form, the H_{CC} region of the Clostridial toxin binding domain is depicted. This region comprises the β-trefoil domain which comprises in an amino to carboxyl linear organization an α-fold, a β4/β5 hairpin turn, a β-fold, a β8/β9 hairpin turn and a γ-fold.

FIG. 3 shows TEM domain organization with a targeting domain located at the amino terminus of a TEM. FIG. 3A depicts the single-chain polypeptide form of a TEM with an amino to carboxyl linear organization comprising a targeting domain, a translocation domain, a di-chain loop region comprising an exogenous protease cleavage site (P), and an enzymatic domain. Upon proteolytic cleavage with a P protease, the single-chain form of the TEM is converted to the di-chain form. FIG. 3B depicts the single polypeptide form of a TEM with an amino to carboxyl linear organization comprising a targeting domain, an enzymatic domain, a di-chain loop region comprising an exogenous protease cleavage site (P), and a translocation domain. Upon proteolytic cleavage with a P protease, the single-chain form of the TEM is converted to the di-chain form.

FIG. 4 shows a TEM domain organization with a targeting domain located between the other two domains. FIG. 4A depicts the single polypeptide form of a TEM with an amino to carboxyl linear organization comprising an enzymatic domain, a di-chain loop region comprising an exogenous protease cleavage site (P), a targeting domain, and a translocation domain. Upon proteolytic cleavage with a P protease, the single-chain form of the TEM is converted to the di-chain form. FIG. 4B depicts the single polypeptide form of a TEM with an amino to carboxyl linear organization comprising a translocation domain, a di-chain loop region comprising an exogenous protease cleavage site (P), a targeting domain, and an enzymatic domain. Upon proteolytic cleavage with a P protease, the single-chain form of the TEM is converted to the di-chain form. FIG. 4C depicts the single polypeptide form of a TEM with an amino to carboxyl linear organization comprising an enzymatic domain, a targeting domain, a di-chain loop region comprising an exogenous protease cleavage site (P), and a translocation domain. Upon proteolytic cleavage with a P protease, the single-chain form of the TEM is converted to the di-chain form. FIG. 4D depicts the single polypeptide form of a TEM with an amino to carboxyl linear organization comprising a translocation domain, a targeting domain, a di-chain loop region comprising an exogenous protease cleavage site (P), and an enzymatic domain. Upon proteolytic cleavage with a P protease, the single-chain form of the TEM is converted to the di-chain form.

FIG. 5 shows a TEM domain organization with a targeting domain located at the carboxyl terminus of the TEM. FIG. 5A depicts the single polypeptide form of a TEM with an amino to carboxyl linear organization comprising an enzymatic domain, a di-chain loop region comprising an exogenous protease cleavage site (P), a translocation domain, and a targeting domain. Upon proteolytic cleavage with a P protease, the single-chain form of the TEM is converted to the di-chain form. FIG. 5B depicts the single polypeptide form of a TEM with an amino to carboxyl linear organization comprising a translocation domain, a di-chain loop region comprising an exogenous protease cleavage site (P), an enzymatic domain, and a targeting domain. Upon proteolytic cleavage with a P protease, the single-chain form of the TEM is converted to the di-chain form.

### DESCRIPTION

Sexual stimulation causes physical changes in a person, most significantly in the sex organs (genital organs). Sexual arousal for a man is usually indicated by penile erection (or penile tumescence). Penile erection results from a complex interaction of physiological, psychological, neural, vascular and endocrine factors, and is usually, though not exclusively, associated with sexual arousal. Clitoral erection occurs when an increase in blood flow fills sinusoids contained in two expandable, tubular structures that run the length of the penis called the corpora cavernosa. The increase in penile blood supply is a result of arterial vasodilatation and smooth muscle relaxation in the corpus cavernosum. This blood engorgement leads to increased penile size and tumescence. The corpus spongiosum is a single tubular structure located just below the corpora cavernosa, which contains the urethra, through which urine and semen pass during urination and ejaculation, respectively. This may also become slightly engorged with blood, but less so than the corpora cavernosa. As such, the failure to achieve penile tumescence may be an important factor in male sexual dysfunction.

The innervation of the penis is both autonomic (sympathetic and parasympathetic) and somatic (sensory and motor). From the neurons in the spinal cord and peripheral ganglia, the sympathetic and parasympathetic nerves merge to form the cavernous nerves, which enter the corpora cavernosa and corpus spongiosum to affect the neurovascular events during tumescence and detumescence. The sympathetic pathway originates from the 11th thoracic to the 2nd lumbar spinal segments and passes through the white rami to the sympathetic chain ganglia. Some fibers then travel through the lumbar splanchnic nerves to the inferior mesenteric and superior hypogastric plexuses, from which fibers travel in the hypogastric nerves to the pelvic plexus. In humans, the T10 to T12 segments are most often the origin of the sympathetic fibers, and the chain ganglia cells projecting to the penis are located in the sacral and caudal ganglia.

The parasympathetic pathway arises from neurons in the intermediolateral cell columns of the second, third, and fourth sacral spinal cord segments. The preganglionic fibers pass in the pelvic nerves to the pelvic plexus, where they are joined by the sympathetic nerves from the superior hypogastric plexus. The cavernous nerves are branches of the pelvic plexus that innervate the penis. Medial branches of the cavernous nerves accompany the urethra the and lateral branches of the cavernous nerves pierce the urogenital diaphragm 4 to 7 mm lateral to the sphincter and form multiple communications between the cavernous and the dorsal nerves. The cavernous nerves are easily damaged during radical excision of the rectum, bladder, and prostate. Other branches of the pelvic plexus innervate the rectum, bladder, prostate, and sphincters.

In the presence of mechanical stimulation, erection is initiated by the parasympathetic division of the autonomic nervous system (ANS) with minimal input from the central nervous system. Parasympathetic branches extend from the sacral plexus into the arteries supplying the erectile tissue; upon stimulation, these nerve branches release acetylcholine, which, in turn causes release of nitric oxide from endothelial cells in the trabecular arteries. Nitric oxide diffuses to the smooth muscle of the arteries (called trabecular smooth muscle), acting as a vasodilating agent. The arteries dilate, filling the corpora spongiosum and cavernosa with blood. The ischiocavernosus and bulbospongiosus muscles also compress the veins of the corpora cavernosa, limiting the venous drainage of blood. Erection subsides when parasympathetic stimulation is discontinued; baseline stimulation from the sympathetic division of the ANS causes constriction of the penile arteries, forcing blood out of the erectile tissue. The cerebral cortex can initiate erection in the absence of direct mechanical stimulation (in response to visual, auditory, olfactory, imagined, or tactile stimuli) acting through erectile centers in the lumbar and sacral regions of the spinal cord. The cortex can suppress erection even in the presence of mechanical stimulation, as can other psychological, emotional, and environmental factors. The opposite term is detumescence (the act of subsiding from a swollen state, especially the relaxation of an erect penis).

Stimulation of the pelvic plexus and the cavernous nerves induces erection, whereas stimulation of the sympathetic trunk causes detumescence. This implies that the sacral parasympathetic input is responsible for tumescence and the thoracolumbar sympathetic pathway is responsible for detumescence. In experiments with cats and rats, removal of the spinal cord below L4 or L5 reportedly eliminated the reflex erectile response but placement with a female in heat or electrical stimulation of the medial preoptic area produced marked erection. Additionally, apomorphine-induced erection appears similar to psychogenic erection in the rat and can be induced by means of the thoracolumbar sympathetic pathway in case of injury to the sacral parasympathetic centers. In man, many individuals with sacral spinal cord injury retain psychogenic erectile ability even though reflexogenic erection is abolished. These cerebrally elicited erections are found more frequently in individuals with lower motoneuron lesions below T12. No psychogenic erection occurs in individuals with lesions above T9; the efferent sympathetic outflow is thus suggested to be at the levels T11 and T12. Also reported, in these individuals with psychogenic erections, lengthening and swelling of the penis are observed but rigidity is insufficient.

It is, therefore, possible that cerebral impulses normally travel through sympathetic (inhibiting norepinephrine release), parasympathetic (releasing NO and acetylcholine), and somatic (releasing acetylcholine) pathways to produce a normal rigid erection. In individuals with a sacral cord lesion, the cerebral impulses can still travel by means of the sympathetic pathway to inhibit norepinephrine release, and NO and acetylcholine can still be released through synapse with postganglionic parasympathetic and somatic neurons. Because the number of synapses between the thoracolumbar outflow and the postganglionic parasympathetic and somatic neurons is less than the sacral outflow, the resulting erection will not be as strong.

The somatic nerves are primarily responsible for sensation and the contraction of the bulbocavernosus and ischiocavernosus muscles. The somatosensory pathway originates at the sensory receptors in the penile skin, glans, and urethra and within the corpus cavernosum. In the human glans penis are numerous afferent terminations: free nerve endings and corpuscular receptors with a ratio of 10:1. The free nerve endings are derived from thin myelinated Aδ and unmyelinated C fibers and are unlike any other cutaneous area in the body. The nerve fibers from the receptors converge to form bundles of the dorsal nerve of the penis, which joins other nerves to become the pudendal nerve. The latter enters the spinal cord via the S2-S4 roots to terminate on spinal neurons and interneurons in the central gray region of the lumbosacral segment. Activation of these sensory neurons sends messages of pain, temperature, and touch by means of spinothalamic and spinoreticular pathways to the thalamus and sensory cortex for sensory perception. The dorsal nerve of the penis used to be regarded as a purely somatic nerve; however, nerve bundles testing positive for nitric oxide synthase (NOS), which is autonomic in origin, have been demonstrated in humans and other mammals. Stimulation of the sympathetic chain at the L4-L5 level elicits an evoked discharge on the dorsal nerve of the penis and stimulation of the dorsal nerve evokes a reflex discharge in the lumbosacral sympathetic chain of rats. These findings clearly demonstrate that the dorsal nerve is a mixed nerve with both somatic and autonomic components that enable it to regulate both erectile and ejaculatory function.

Onuf's nucleus in the second to fourth sacral spinal segments is the center of somatomotor penile innervation. These nerves travel in the sacral nerves to the pudendal nerve to innervate the ischiocavernosus and bulbocavernosus muscles. Contraction of the ischiocavernosus muscles produces the rigid-erection phase. Rhythmic contraction of the bulbocavernosus muscle is necessary for ejaculation. In animal studies, direct innervation of the sacral spinal motoneurons by brain stem sympathetic centers (A5-catecholaminergic cell group and locus coeruleus) has been identified. This adrenergic innervation of pudendal motoneurons may be involved in rhythmic contractions of perineal muscles during ejaculation. In addition, oxytocinergic and serotonergic innervation of lumbosacral nuclei controlling penile erection and perineal muscles in the male rat has also been demonstrated. Depending on the intensity and nature of genital stimulation, several spinal reflexes can be elicited by stimulation of the genitalia. The best known is the bulbocavernosus reflex, which is the basis of genital neurologic examination and electrophysiologic latency testing. Impairment of bulbocavernosus and ischiocavernosus muscles appears to impair penile erection.

Studies in animals have identified the medial preoptic area (MPOA) and the paraventricular nucleus (PVN) of the hypothalamus and hippocampus as important integration centers for sexual function and penile erection: electrostimulation of this area induces erection, and lesions at this site limit copulation. Stimulation of the dorsal nerve of the penis in the rat influenced the firing rate of about 80% of the neurons in the MPOA but not in other areas of the hypothalamus. Efferent pathways from the MPOA enter the medial forebrain bundle and the midbrain tegmental region (near the substantia nigra). Pathologic processes in these regions, such as Parkinson's disease or cerebrovascular accidents, are often associated with erectile dysfunction. Axonal tracing in monkeys, cats and rats has shown direct projection from hypothalamic nuclei to the lumbosacral autonomic erection centers. The neurons in these hypothalamic nuclei contain peptidergic neurotransmitters, including oxytocin and vasopressin, which may be involved in penile erection. Several brain stem and medullary centers are also involved in sexual function. The A5 catecholamine cell group and locus ceruleus have been shown to provide adrenergic innervation to hypothalamus, thalamus, neocortex and spinal cord. Projections from the nucleus paragigantocellularis, which provides inhibitory serotonergic innervation, have also been demonstrated in hypothalamus, the limbic system, the neocortex and the spinal cord.

The homologous organ to the male penis in a female is the clitoris. This organ varies in size from a few millimeters to one centimeter, is located in the upper labial fold, and includes an external short head attached to a long body which is internally located. The body of the clitoris is surrounded by bulky erectile tissue on either side which is composed of muscular trabeculae with potential vascular spaces that admit blood from arterioles during engorgement. The clitoris is richly innervated with sensory nerves. The major nerve which produces sensations to the clitoris is a branch of the pudendal nerve, also known as the dorsal nerve of the clitoris.

Clitoral erection (of clitoral tumescence) results from a complex interaction of physiological, psychological, neural, vascular and endocrine factors, and is usually, though not exclusively, associated with sexual arousal. Clitoral erection occurs when an increase in blood flow supplied by the dorsal clitoral and cavernosal clitoral arteries fills sinusoids contained in two expandable erectile structures called the corpora cavernosa. As in males, the increase in clitoral blood supply is a result of arterial vasodilatation and smooth muscle relaxation in the corpus cavernosum. This blood engorgement leads to increased clitoral size and tumescence. Clitoral tumescence results in extrusion of the glans clitoridis and thinning of the skin enhances sensitivity to physical stimulation. Physical stimulation of the tumescent clitoris provokes pelvic floor muscle contraction, which triggers the female orgasm. After a female has orgasmed, the clitoral erection usually ends (detumescence), but this may take time. Clitoral tumescence also results in increased blood supply to the vulva, vaginal transudation (secretion of moisture through the vaginal walls which serves as lubrication), nipple erection, and prolonged body relaxation. As such, the failure to achieve clitoral tumescence may be an important factor in female sexual dysfunction.

Clostridia toxins produced by Clostridium botulinum, Clostridium tetani, Clostridium baratii and Clostridium butyricum are the most widely used in therapeutic and cosmetic treatments of humans and other mammals. Strains of C. botulinum produce seven antigenically-distinct types of Botulinum toxins (BoNTs), which have been identified by investigating botulism outbreaks in man (BoNT/A, BoNT/B, BoNT/E and BoNT/F), animals (BoNT/C1 and BoNT/D), or isolated from soil (BoNT/G). BoNTs possess approximately 35% amino acid identity with each other and share the same functional domain organization and overall structural architecture. It is recognized by those of skill in the art that within each type of Clostridial toxin there can be subtypes that differ somewhat in their amino acid sequence, and also in the nucleic acids encoding these proteins. For example, there are presently five BoNT/A subtypes, BoNT/A1, BoNT/A2, BoNT/A3 BoNT/A4 and BoNT/A5, with specific subtypes showing approximately 89% amino acid identity when compared to another BoNT/A subtype. While all seven BoNT serotypes have similar structure and pharmacological properties, each also displays heterogeneous bacteriological characteristics. In contrast, tetanus toxin (TeNT) is produced by a uniform group of C. tetani. Two other Clostridia species, C. baratii and C. butyricum, produce toxins, BaNT and BuNT, which are functionally similar to BoNT/F and BoNT/E, respectively.

Clostridial toxins are released by Clostridial bacterium as complexes comprising the approximately 150-kDa Clostridial toxin along with associated non-toxin proteins (NAPs). Identified NAPs include proteins possessing hemaglutination activity, such, e.g., a hemagglutinin of approximately 17-kDa (HA-17), a hemagglutinin of approximately 33-kDa (HA-33) and a hemagglutinin of approximately 70-kDa (HA-70); as well as non-toxic non-hemagglutinin (NTNH), a protein of approximately 130-kDa. Thus, the botulinum toxin type A complex can be produced by Clostridial bacterium as 900-kDa, 500-kDa and 300-kDa forms. Botulinum toxin types B and C₁ are apparently produced as only a 500-kDa complex. Botulinum toxin type D is produced as both 300-kDa and 500-kDa complexes. Finally, botulinum toxin types E and F are produced as only approximately 300-kDa complexes. The differences in molecular weight for the complexes are due to differing ratios of NAPs. The toxin complex is important for the intoxication process because it provides protection from adverse environmental conditions, resistance to protease digestion, and appears to facilitate internalization and activation of the toxin.

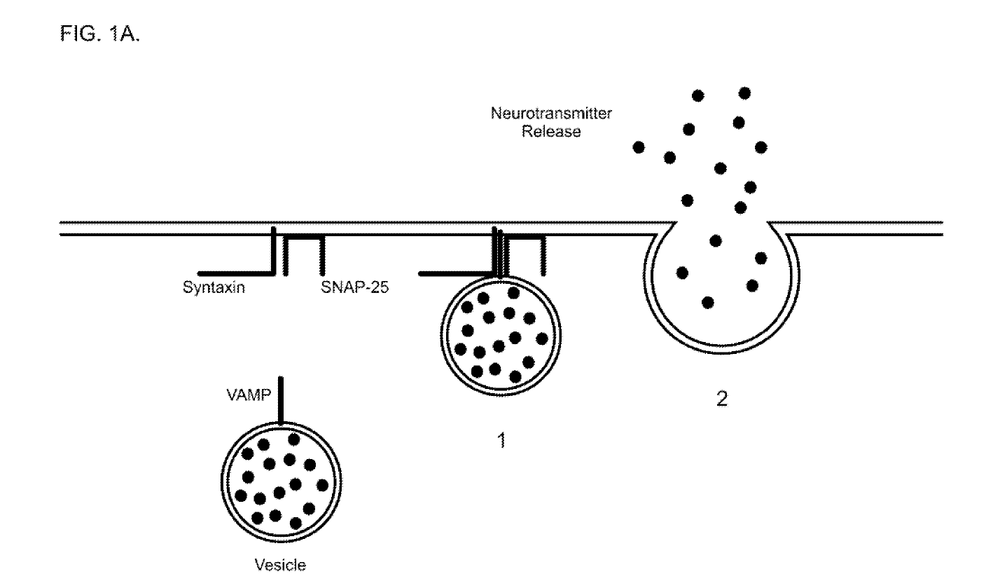

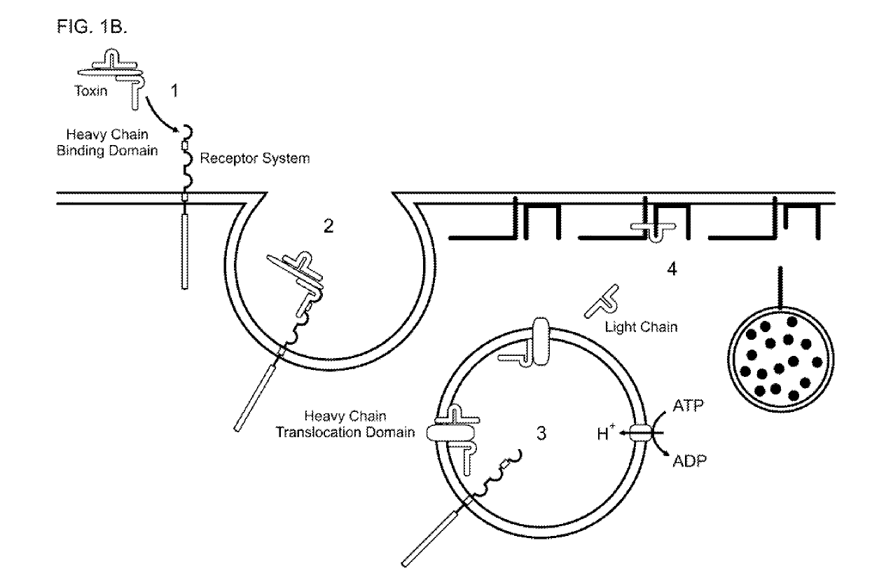

A Clostridial toxin itself is translated as a single chain polypeptide that is subsequently cleaved by proteolytic scission within a disulfide loop by a naturally-occurring protease (FIG. 1). This cleavage occurs within the discrete di-chain loop region created between two cysteine residues that form a disulfide bridge. This posttranslational processing yields a di-chain molecule comprising an approximately 50 kDa light chain (LC) and an approximately 100 kDa heavy chain (HC) held together by the single disulfide bond and non-covalent interactions between the two chains. The naturally-occurring protease used to convert the single chain molecule into the di-chain is currently not known. In some serotypes, such as, e.g., BoNT/A, the naturally-occurring protease is produced endogenously by the bacteria serotype and cleavage occurs within the cell before the toxin is release into the environment. However, in other serotypes, such as, e.g., BoNT/E, the bacterial strain appears not to produce an endogenous protease capable of converting the single chain form of the toxin into the di-chain form. In these situations, the toxin is released from the cell as a single-chain toxin which is subsequently converted into the di-chain form by a naturally-occurring protease found in the environment.

Each mature di-chain molecule of a Clostridial toxin comprises three functionally distinct domains: 1) an enzymatic domain located in the light chain (LC) that includes a metalloprotease region containing a zinc-dependent endopeptidase activity which specifically targets core components of the neurotransmitter release apparatus; 2) a translocation domain contained within the amino-terminal half of the heavy chain (H_{N}) that facilitates release of the LC from intracellular vesicles into the cytoplasm of the target cell; and 3) a binding domain found within the carboxyl-terminal half of the heavy chain (H_{C}) that determines the binding activity and binding specificity of the toxin to the receptor complex located at the surface of the target cell. The H_{C} domain comprises two distinct structural features of roughly equal size that indicate function and are designated the H_{CN} and H_{CC} subdomains.

Clostridial toxins act on the nervous system by blocking the release of acetylcholine (ACh) at the pre-synaptic neuromuscular junction. The binding, translocation and enzymatic activity of these three functional domains are all necessary for toxicity. While all details of this process are not yet precisely known, the overall cellular intoxication mechanism whereby Clostridial toxins enter a neuron and inhibit neurotransmitter release is similar, regardless of serotype or subtype. Although applicants have no wish to be limited by the following description, the intoxication mechanism can be described as comprising at least four steps: 1) receptor binding, 2) complex internalization, 3) light chain translocation, and 4) enzymatic target modification (FIG. 1). The process is initiated when the binding domain of a Clostridial toxin binds to a toxin-specific receptor system located on the plasma membrane surface of a target cell. The binding specificity of a receptor complex is thought to be achieved, in part, by specific combinations of gangliosides and protein receptors that appear to distinctly comprise each Clostridial toxin receptor complex. Once bound, the toxin/receptor complexes are internalized by endocytosis and the internalized vesicles are sorted to specific intracellular routes. The translocation step appears to be triggered by the acidification of the vesicle compartment. This process seems to initiate pH-dependent structural rearrangements that increase hydrophobicity, create a pore in the vesicle membrane, and promote formation of the di-chain form of the toxin. Once di-chain formation occurs, light chain endopeptidase of the toxin is released from the intracellular vesicle via the pore into the cytosol where it appears to specifically target one of three known core components of the neurotransmitter release apparatus. These core proteins, vesicle-associated membrane protein (VAMP)/synaptobrevin, synaptosomal-associated protein of 25 kDa (SNAP-25) and Syntaxin, are necessary for synaptic vesicle docking and fusion at the nerve terminal and constitute members of the soluble N-ethylmaleimide-sensitive factor-attachment protein-receptor (SNARE) family. BoNT/A and BoNT/E cleave SNAP-25 in the carboxyl-terminal region, releasing a nine or twenty-six amino acid segment, respectively, and BoNT/C1 also cleaves SNAP-25 near the carboxyl-terminus. The botulinum serotypes BoNT/B, BoNT/D, BoNT/F and BoNT/G, and tetanus toxin, act on the conserved central portion of VAMP, and release the amino-terminal portion of VAMP into the cytosol. BoNT/C1 cleaves syntaxin at a single site near the cytosolic membrane surface.

Aspects of the present specification disclose, in part, in part, a Clostridial toxin. As used herein, the term “Clostridial toxin” refers to any toxin produced by a Clostridial toxin strain that can execute the overall cellular mechanism whereby a Clostridial toxin intoxicates a cell and encompasses the binding of a Clostridial toxin to a low or high affinity Clostridial toxin receptor, the internalization of the toxin/receptor complex, the translocation of the Clostridial toxin light chain into the cytoplasm and the enzymatic modification of a Clostridial toxin substrate. Non-limiting examples of Clostridial toxins include a Botulinum toxin like BoNT/A, a BoNT/B, a BoNT/C₁, a BoNT/D, a BoNT/E, a BoNT/F, a BoNT/G, a Tetanus toxin (TeNT), a Baratii toxin (BaNT), and a Butyricum toxin (BuNT). The BoNT/C₂ cytotoxin and BoNT/C₃ cytotoxin, not being neurotoxins, are excluded from the term “Clostridial toxin.” A Clostridial toxin disclosed herein includes, without limitation, naturally occurring Clostridial toxin variants, such as, e.g., Clostridial toxin isoforms and Clostridial toxin subtypes; non-naturally occurring Clostridial toxin variants, such as, e.g., conservative Clostridial toxin variants, non-conservative Clostridial toxin variants, Clostridial toxin chimeric variants and active Clostridial toxin fragments thereof, or any combination thereof.

A Clostridial toxin disclosed herein also includes a Clostridial toxin complex. As used herein, the term “Clostridial toxin complex” refers to a complex comprising a Clostridial toxin and non-toxin associated proteins (NAPs), such as, e.g., a Botulinum toxin complex, a Tetanus toxin complex, a Baratii toxin complex, and a Butyricum toxin complex. Non-limiting examples of Clostridial toxin complexes include those produced by a Clostridium botulinum, such as, e.g., a 900-kDa BoNT/A complex, a 500-kDa BoNT/A complex, a 300-kDa BoNT/A complex, a 500-kDa BoNT/B complex, a 500-kDa BoNT/C₁ complex, a 500-kDa BoNT/D complex, a 300-kDa BoNT/D complex, a 300-kDa BoNT/E complex, and a 300-kDa BoNT/F complex.

Clostridial toxins can be produced using standard purification or recombinant biology techniques known to those skilled in the art. See, e.g., Hui Xiang et al., Animal Product Free System and Process for Purifying a Botulinum Toxin, U.S. Pat. No. 7,354,740, which is hereby incorporated by reference in its entirety. For example, a BoNT/A complex can be isolated and purified from an anaerobic fermentation by cultivating Clostridium botulinum type A in a suitable medium. Raw toxin can be harvested by precipitation with sulfuric acid and concentrated by ultramicrofiltration. Purification can be carried out by dissolving the acid precipitate in calcium chloride. The toxin can then be precipitated with cold ethanol. The precipitate can be dissolved in sodium phosphate buffer and centrifuged. Upon drying there can then be obtained approximately 900 kD crystalline BoNT/A complex with a specific potency of 3×10⁷ LD₅₀ U/mg or greater. Furthermore, NAPs can be separated out to obtain purified toxin, such as e.g., BoNT/A with an approximately 150 kD molecular weight with a specific potency of 1-2×10⁸ LD₅₀ U/mg or greater, purified BoNT/B with an approximately 156 kD molecular weight with a specific potency of 1-2×10⁸ LD₅₀ U/mg or greater, and purified BoNT/F with an approximately 155 kD molecular weight with a specific potency of 1-2×10⁷ LD₅₀ U/mg or greater. See Edward J. Schantz & Eric A. Johnson, Properties and use of Botulinum Toxin and Other Microbial Neurotoxins in Medicine, Microbiol Rev. 56: 80-99 (1992), which is hereby incorporated in its entirety. As another example, recombinant Clostridial toxins can be recombinantly produced as described in Steward et al., Optimizing Expression of Active Botulinum Toxin Type A, U.S. Patent Publication 2008/0057575; and Steward et al., Optimizing Expression of Active Botulinum Toxin Type E, U.S. Patent Publication 2008/0138893, each of which is hereby incorporated in its entirety.

Clostridial toxins are also commercially available as pharmaceutical compositions include, BoNT/A preparations, such as, e.g., BOTOX® (Allergan, Inc., Irvine, Calif.), DYSPORT®/RELOXIN®, (Beaufour Ipsen, Porton Down, England), NEURONOX® (Medy-Tox, Inc., Ochang-myeon, South Korea), BTX-A (Lanzhou Institute Biological Products, China) and XEOMIN® (Merz Pharmaceuticals, GmbH., Frankfurt, Germany); and BoNT/B preparations, such as, e.g., MYOBLOC™/NEUROBLOC™ (Solstice Neurosciences, Inc., South San Francisco, Calif.). Clostridial toxin complexes may be obtained from, e.g., List Biological Laboratories, Inc. (Campbell, Calif.), the Centre for Applied Microbiology and Research (Porton Down, U.K), Wako (Osaka, Japan), and Sigma Chemicals (St Louis, Mo.).

In an embodiment, a Clostridial may be a Botulinum toxin, Tetanus toxin, a Baratii toxin, or a Butyricum toxin. In aspects of this embodiment, a Botulinum toxin may be a BoNT/A, a BoNT/B, a BoNT/C₁, a BoNT/D, a BoNT/E, a BoNT/F, or a BoNT/G. In another embodiment, a Clostridial toxin may be a Clostridial toxin variant. In aspects of this embodiment, a Clostridial toxin variant may be a naturally-occurring Clostridial toxin variant or a non-naturally-occurring Clostridial toxin variant. In other aspects of this embodiment, a Clostridial toxin variant may be a BoNT/A variant, a BoNT/B variant, a BoNT/C₁ variant, a BoNT/D variant, a BoNT/E variant, a BoNT/F variant, a BoNT/G variant, a TeNT variant, a BaNT variant, or a BuNT variant, where the variant is either a naturally-occurring variant or a non-naturally-occurring variant.

In an embodiment, a Clostridial toxin may be a Clostridial toxin complex. In aspects of this embodiment, a Clostridial toxin complex may be a BoNT/A complex, a BoNT/B complex, a BoNT/C₁ complex, a BoNT/D complex, a BoNT/E complex, a BoNT/F complex, a BoNT/G complex, a TeNT complex, a BaNT complex, or a BuNT complex. In other aspects of this embodiment, a Clostridial toxin complex may be a 900-kDa BoNT/A complex, a 500-kDa BoNT/A complex, a 300-kDa BoNT/A complex, a 500-kDa BoNT/B complex, a 500-kDa BoNT/C1 complex, a 500-kDa BoNT/D complex, a 300-kDa BoNT/D complex, a 300-kDa BoNT/E complex, or a 300-kDa BoNT/F complex.

Aspects of the present disclosure comprise, in part, a Targeted Exocytosis Modulator. As used herein, the term “Targeted Exocytosis Modulator” is synonymous with “TEM” or “retargeted endopeptidase.” Generally, a TEM comprises an enzymatic domain from a Clostridial toxin light chain, a translocation domain from a Clostridial toxin heavy chain, and a targeting domain. The targeting domain of a TEM provides an altered cell targeting capability that targets the molecule to a receptor other than the native Clostridial toxin receptor utilized by a naturally-occurring Clostridial toxin. This re-targeted capability is achieved by replacing the naturally-occurring binding domain of a Clostridial toxin with a targeting domain having a binding activity for a non-Clostridial toxin receptor. Although binding to a non-Clostridial toxin receptor, a TEM undergoes all the other steps of the intoxication process including internalization of the TEM/receptor complex into the cytoplasm, formation of the pore in the vesicle membrane and di-chain molecule, translocation of the enzymatic domain into the cytoplasm, and exerting a proteolytic effect on a component of the SNARE complex of the target cell.

However, an important difference between TEMs, such as, e.g., TEMs disclosed herein, and native Clostridial toxins is that since TEMs do not target motor neurons, the lethality associated with over-dosing an individual with a TEM is greatly minimized, if not avoided altogether. For example, a TEM comprising an opioid targeting domain can be administered at 10,000 times the therapeutically effective dose before evidence of lethality is observed, and this lethality is due to the passive diffusion of the molecule and not via the intoxication process. Thus, for all practical purposes TEMs are non-lethal molecules.

As used herein, the term “Clostridial toxin enzymatic domain” refers to a Clostridial toxin polypeptide located in the light chain of a Clostridial toxin that executes the enzymatic target modification step of the intoxication process. A Clostridial toxin enzymatic domain includes a metalloprotease region containing a zinc-dependent endopeptidase activity which specifically targets core components of the neurotransmitter release apparatus. Thus, a Clostridial toxin enzymatic domain specifically targets and proteolytically cleavages of a Clostridial toxin substrate, such as, e.g., SNARE proteins like a SNAP-25 substrate, a VAMP substrate and a Syntaxin substrate.

A Clostridial toxin enzymatic domain includes, without limitation, naturally occurring Clostridial toxin enzymatic domain variants, such as, e.g., Clostridial toxin enzymatic domain isoforms and Clostridial toxin enzymatic domain subtypes; non-naturally occurring Clostridial toxin enzymatic domain variants, such as, e.g., conservative Clostridial toxin enzymatic domain variants, non-conservative Clostridial toxin enzymatic domain variants, Clostridial toxin enzymatic domain chimeras, active Clostridial toxin enzymatic domain fragments thereof, or any combination thereof. Non-limiting examples of a Clostridial toxin enzymatic domain include, e.g., a BoNT/A enzymatic domain, a BoNT/B enzymatic domain, a BoNT/C1 enzymatic domain, a BoNT/D enzymatic domain, a BoNT/E enzymatic domain, a BoNT/F enzymatic domain, a BoNT/G enzymatic domain, a TeNT enzymatic domain, a BaNT enzymatic domain, and a BuNT enzymatic domain.

As used herein, the term “Clostridial toxin translocation domain” refers to a Clostridial toxin polypeptide located within the amino-terminal half of the heavy chain of a Clostridial toxin that executes the translocation step of the intoxication process. The translocation step appears to involve an allosteric conformational change of the translocation domain caused by a decrease in pH within the intracellular vesicle. This conformational change results in the formation of a pore in the vesicular membrane that permits the movement of the light chain from within the vesicle into the cytoplasm. Thus, a Clostridial toxin translocation domain facilitates the movement of a Clostridial toxin light chain across a membrane of an intracellular vesicle into the cytoplasm of a cell.

A Clostridial toxin translocation domain includes, without limitation, naturally occurring Clostridial toxin translocation domain variants, such as, e.g., Clostridial toxin translocation domain isoforms and Clostridial toxin translocation domain subtypes; non-naturally occurring Clostridial toxin translocation domain variants, such as, e.g., conservative Clostridial toxin translocation domain variants, non-conservative Clostridial toxin translocation domain variants, Clostridial toxin translocation domain chimerics, active Clostridial toxin translocation domain fragments thereof, or any combination thereof. Non-limiting examples of a Clostridial toxin translocation domain include, e.g., a BoNT/A translocation domain, a BoNT/B translocation domain, a BoNT/C1 translocation domain, a BoNT/D translocation domain, a BoNT/E translocation domain, a BoNT/F translocation domain, a BoNT/G translocation domain, a TeNT translocation domain, a BaNT translocation domain, and a BuNT translocation domain.

As used herein, the term “targeting domain” is synonymous with “binding domain” or “targeting moiety” and refers to a polypeptide that executes the receptor binding and/or complex internalization steps of the intoxication process, with the proviso that the binding domain is not a Clostridial toxin binding domain found within the carboxyl-terminal half of the heavy chain of a Clostridial toxin. A targeting domain includes a receptor binding region that confers the binding activity and/or specificity of the targeting domain for its cognate receptor. As used herein, the term “cognate receptor” refers to a receptor for which the targeting domain preferentially interacts with under physiological conditions, or under in vitro conditions substantially approximating physiological conditions. As used herein, the term “preferentially interacts” is synonymous with “preferentially binding” and refers to an interaction that is statistically significantly greater in degree relative to a control. With reference to a targeting domain disclosed herein, a targeting domain binds to its cognate receptor to a statistically significantly greater degree relative to a non-cognate receptor. Said another way, there is a discriminatory binding of the targeting domain to its cognate receptor relative to a non-cognate receptor. Thus, a targeting domain directs binding to a TEM-specific receptor located on the plasma membrane surface of a target cell.

In an embodiment, a targeting domain disclosed herein has an association rate constant that confers preferential binding to its cognate receptor. In aspects of this embodiment, a targeting domain disclosed herein binds to its cognate receptor with an association rate constant of, e.g., less than 1×10⁵ M⁻¹ s⁻¹, less than 1×10⁶ M⁻¹ s⁻¹, less than 1×10⁷ M⁻¹ s⁻¹, or less than 1×10⁸ M⁻¹ s⁻¹. In other aspects of this embodiment, a targeting domain disclosed herein binds to its cognate receptor with an association rate constant of, e.g., more than 1×10⁵ M⁻¹ s⁻¹, more than 1×10⁶ M⁻¹ s⁻¹, more than 1×10⁷ M⁻¹ s⁻¹ or more than 1×10⁸ M⁻¹ s⁻¹. In yet other aspects of this embodiment, a targeting domain disclosed herein binds to its cognate receptor with an association rate constant between 1×10⁵ M⁻¹ s⁻¹ to 1×10⁸ M⁻¹ s⁻¹, 1×10⁶ M⁻¹ s⁻¹ to 1×10⁸ M⁻¹ s⁻¹, 1×10⁵ M⁻¹ s⁻¹ to 1×10⁷ M⁻¹ s⁻¹, or 1×10⁶ M⁻¹ s⁻¹ to 1×10⁷ M⁻¹ s⁻¹.

In another embodiment, a targeting domain disclosed herein has an association rate constant that is greater for its cognate target receptor relative to a non-cognate receptor. In other aspects of this embodiment, a targeting domain disclosed herein has an association rate constant that is greater for its cognate target receptor relative to a non-cognate receptor by, at least one-fold, at least two-fold, at least three-fold, at least four fold, at least five-fold, at least 10 fold, at least 50 fold, at least 100 fold, at least 1000 fold, at least 10,000 fold, or at least 100,000 fold. In other aspects of this embodiment, a targeting domain disclosed herein has an association rate constant that is greater for its cognate target receptor relative to a non-cognate receptor by, e.g., about one-fold to about three-fold, about one-fold to about five-fold, about one-fold to about 10-fold, about one-fold to about 100-fold, about one-fold to about 1000-fold, about five-fold to about 10-fold, about five-fold to about 100-fold, about five-fold to about 1000-fold, about 10-fold to about 100-fold, about 10-fold to about 1000-fold, about 10-fold to about 10.000-fold, or about 10-fold to about 100.000-fold.

In yet another embodiment, a targeting domain disclosed herein has a disassociation rate constant that confers preferential binding to its cognate receptor. In other aspects of this embodiment, a targeting domain disclosed herein binds to its cognate receptor with a disassociation rate constant of less than 1×10⁻³ s⁻¹, less than 1×10⁻⁴ s⁻¹, or less than 1×10⁻⁵ s⁻¹. In yet other aspects of this embodiment, a targeting domain disclosed herein binds to its cognate receptor with a disassociation rate constant of, e.g., less than 1.0×10⁻⁴ s⁻¹, less than 2.0×10⁻⁴ s⁻¹, less than 3.0×10⁻⁴ s⁻¹, less than 4.0×10⁻⁴ s⁻¹, less than 5.0×10⁻⁴ s⁻¹, less than 6.0×10⁻⁴ s⁻¹, less than 7.0×10⁻⁴ s⁻¹, less than 8.0×10⁻⁴ s⁻¹, or less than 9.0×10⁻⁴ s⁻¹. In still other aspects of this embodiment, a targeting domain disclosed herein binds to its cognate receptor with a disassociation rate constant of, e.g., more than 1×10⁻³ s⁻¹, more than 1×10⁻⁴ s⁻¹, or more than 1×10⁻⁵ s⁻¹. In other aspects of this embodiment, a targeting domain disclosed herein binds to its cognate receptor with a disassociation rate constant of, e.g., more than 1.0×10⁻⁴ s⁻¹, more than 2.0×10⁻⁴ s⁻¹, more than 3.0×10⁻⁴ s⁻¹, more than 4.0×10⁻⁴ s⁻¹, more than 5.0×10⁻⁴ s⁻¹, more than 6.0×10⁻⁴ s⁻¹, more than 7.0×10⁻⁴ s⁻¹, more than 8.0×10⁻⁴ s⁻¹, or more than 90×10⁻⁴ s⁻¹.

In still another embodiment, a targeting domain disclosed herein has a disassociation rate constant that is less for its cognate target receptor relative to a non-cognate receptor. In other aspects of this embodiment, a targeting domain disclosed herein has a disassociation rate constant that is less for its cognate target receptor relative to a non-cognate receptor by, e.g., at least one-fold, at least two-fold, at least three-fold, at least four fold, at least five-fold, at least 10 fold, at least 50 fold, at least 100 fold, at least 1000 fold, at least 10,000 fold, or at least 100,000 fold. In other aspects of this embodiment, a targeting domain disclosed herein has a disassociation rate constant that is less for its cognate target receptor relative to a non-cognate receptor by, e.g., about one-fold to about three-fold, about one-fold to about five-fold, about one-fold to about 10-fold, about one-fold to about 100-fold, about one-fold to about 1000-fold, about five-fold to about 10-fold, about five-fold to about 100-fold, about five-fold to about 1000-fold, about 10-fold to about 100-fold, about 10-fold to about 1000-fold, about 10-fold to about 10.000-fold, or about 10-fold to about 100.000-fold.

In another embodiment, a targeting domain disclosed herein has an equilibrium disassociation constant that confers preferential binding to its cognate receptor. In other aspects of this embodiment, a targeting domain disclosed herein binds to its cognate receptor with an equilibrium disassociation constant of, e.g., less than 0.500 nM. In yet other aspects of this embodiment, a targeting domain disclosed herein binds to its cognate receptor with an equilibrium disassociation constant of, e.g., less than 0.500 nM, less than 0.450 nM, less than 0.400 nM, less than 0.350 nM, less than 0.300 nM, less than 0.250 nM, less than 0.200 nM, less than 0.150 nM, less than 0.100 nM, or less than 0.050 nM. In other aspects of this embodiment, a targeting domain disclosed herein binds to its cognate receptor with an equilibrium disassociation constant of, e.g., more than 0.500 nM, more than 0.450 nM, more than 0.400 nM, more than 0.350 nM, more than 0.300 nM, more than 0.250 nM, more than 0.200 nM, more than 0.150 nM, more than 0.100 nM, or more than 0.050 nM.

In yet another embodiment, a targeting domain disclosed herein has an equilibrium disassociation constant that is greater for its cognate target receptor relative to a non-cognate receptor. In other aspects of this embodiment, a targeting domain disclosed herein has an equilibrium disassociation constant that is greater for its cognate target receptor relative to a non-cognate receptor by, e.g., at least one-fold, at least two-fold, at least three-fold, at least four fold, at least five-fold, at least 10 fold, at least 50 fold, at least 100 fold, at least 1000 fold, at least 10,000 fold, or at least 100,000 fold. In other aspects of this embodiment, a targeting domain disclosed herein has an equilibrium disassociation constant that is greater for its cognate target receptor relative to a non-cognate receptor by, e.g., about one-fold to about three-fold, about one-fold to about five-fold, about one-fold to about 10-fold, about one-fold to about 100-fold, about one-fold to about 1000-fold, about five-fold to about 10-fold, about five-fold to about 100-fold, about five-fold to about 1000-fold, about 10-fold to about 100-fold, about 10-fold to about 1000-fold, about 10-fold to about 10.000-fold, or about 10-fold to about 100.000-fold.

In another embodiment, a targeting domain disclosed herein may be one that preferentially interacts with a receptor located on a sensory neuron. In an aspect of this embodiment, the sensory neuron targeting domain is one whose cognate receptor is located exclusively on the plasma membrane of sensory neurons. In another aspect of this embodiment, the sensory neuron targeting domain is one whose cognate receptor is located primarily on the plasma membrane of sensory neuron. For example, a receptor for a sensory neuron targeting domain is located primarily on a sensory neuron when, e.g., at least 60% of all cells that have a cognate receptor for a sensory neuron targeting domain on the surface of the plasma membrane are sensory neurons, at least 70% of all cells that have a cognate receptor for a sensory neuron targeting domain on the surface of the plasma membrane are sensory neurons, at least 80% of all cells that have a cognate receptor for a sensory neuron targeting domain on the surface of the plasma membrane are sensory neurons, or at least 90% of all cells that have a cognate receptor for a sensory neuron targeting domain on the surface of the plasma membrane are sensory neurons. In yet another aspect of this embodiment, the sensory neuron targeting domain is one whose cognate receptor is located on the plasma membrane of several types of cells, including sensory neurons. In still another aspect of this embodiment, the sensory neuron targeting domain is one whose cognate receptor is located on the plasma membrane of several types of cells, including sensory neurons, with the proviso that motor neurons are not one of the other types of cells.

In another embodiment, a targeting domain disclosed herein may be one that preferentially interacts with a receptor located on a sympathetic neuron. In an aspect of this embodiment, the sympathetic neuron targeting domain is one whose cognate receptor is located exclusively on the plasma membrane of sympathetic neurons. In another aspect of this embodiment, the sympathetic neuron targeting domain is one whose cognate receptor is located primarily on the plasma membrane of sympathetic neuron. For example, a receptor for a sympathetic neuron targeting domain is located primarily on a sympathetic neuron when, e.g., at least 60% of all cells that have a cognate receptor for a sympathetic neuron targeting domain on the surface of the plasma membrane are sympathetic neurons, at least 70% of all cells that have a cognate receptor for a sympathetic neuron targeting domain on the surface of the plasma membrane are sympathetic neurons, at least 80% of all cells that have a cognate receptor for a sympathetic neuron targeting domain on the surface of the plasma membrane are sympathetic neurons, or at least 90% of all cells that have a cognate receptor for a sympathetic neuron targeting domain on the surface of the plasma membrane are sympathetic neurons. In yet another aspect of this embodiment, the sympathetic neuron targeting domain is one whose cognate receptor is located on the plasma membrane of several types of cells, including sympathetic neurons. In still another aspect of this embodiment, the sympathetic neuron targeting domain is one whose cognate receptor is located on the plasma membrane of several types of cells, including sympathetic neurons, with the proviso that motor neurons are not one of the other types of cells.

In another embodiment, a targeting domain disclosed herein may be one that preferentially interacts with a receptor located on a parasympathetic neuron. In an aspect of this embodiment, the parasympathetic neuron targeting domain is one whose cognate receptor is located exclusively on the plasma membrane of parasympathetic neurons. In another aspect of this embodiment, the parasympathetic neuron targeting domain is one whose cognate receptor is located primarily on the plasma membrane of parasympathetic neuron. For example, a receptor for a parasympathetic neuron targeting domain is located primarily on a parasympathetic neuron when, e.g., at least 60% of all cells that have a cognate receptor for a parasympathetic neuron targeting domain on the surface of the plasma membrane are parasympathetic neurons, at least 70% of all cells that have a cognate receptor for a parasympathetic neuron targeting domain on the surface of the plasma membrane are parasympathetic neurons, at least 80% of all cells that have a cognate receptor for a parasympathetic neuron targeting domain on the surface of the plasma membrane are parasympathetic neurons, or at least 90% of all cells that have a cognate receptor for a parasympathetic neuron targeting domain on the surface of the plasma membrane are parasympathetic neurons. In yet another aspect of this embodiment, the parasympathetic neuron targeting domain is one whose cognate receptor is located on the plasma membrane of several types of cells, including parasympathetic neurons. In still another aspect of this embodiment, the parasympathetic neuron targeting domain is one whose cognate receptor is located on the plasma membrane of several types of cells, including parasympathetic neurons, with the proviso that motor neurons are not one of the other types of cells.

In another embodiment, a targeting domain disclosed herein is an opioid peptide targeting domain, a galanin peptide targeting domain, a PAR peptide targeting domain, a somatostatin peptide targeting domain, a neurotensin peptide targeting domain, a SLURP peptide targeting domain, an angiotensin peptide targeting domain, a tachykinin peptide targeting domain, a Neuropeptide Y related peptide targeting domain, a kinin peptide targeting domain, a melanocortin peptide targeting domain, or a granin peptide targeting domain, a glucagon like hormone peptide targeting domain, a secretin peptide targeting domain, a pituitary adenylate cyclase activating peptide (PACAP) peptide targeting domain, a growth hormone-releasing hormone (GHRH) peptide targeting domain, a vasoactive intestinal peptide (VIP) peptide targeting domain, a gastric inhibitory peptide (GIP) peptide targeting domain, a calcitonin peptide targeting domain, a visceral gut peptide targeting domain, a neurotrophin peptide targeting domain, a head activator (HA) peptide, a glial cell line-derived neurotrophic factor (GDNF) family of ligands (GFL) peptide targeting domain, a RF-amide related peptide (RFRP) peptide targeting domain, a neurohormone peptide targeting domain, or a neuroregulatory cytokine peptide targeting domain, an interleukin (IL) targeting domain, vascular endothelial growth factor (VEGF) targeting domain, an insulin-like growth factor (IGF) targeting domain, an epidermal growth factor (EGF) targeting domain, a Transformation Growth Factor-β (TGFβ) targeting domain, a Bone Morphogenetic Protein (BMP) targeting domain, a Growth and Differentiation Factor (GDF) targeting domain, an activin targeting domain, or a Fibroblast Growth Factor (FGF) targeting domain, or a Platelet-Derived Growth Factor (PDGF) targeting domain.

In an aspect of this embodiment, an opioid peptide targeting domain is an enkephalin peptide, a bovine adrenomedullary-22 (BAM22) peptide, an endomorphin peptide, an endorphin peptide, a dynorphin peptide, a nociceptin peptide, or a hemorphin peptide. In another aspect of this embodiment, an enkephalin peptide targeting domain is a Leu-enkephalin peptide, a Met-enkephalin peptide, a Met-enkephalin MRGL peptide, or a Met-enkephalin MRF peptide. In another aspect of this embodiment, a bovine adrenomedullary-22 peptide targeting domain is a BAM22 (1-12) peptide, a BAM22 (6-22) peptide, a BAM22 (8-22) peptide, or a BAM22 (1-22) peptide. In another aspect of this embodiment, an endomorphin peptide targeting domain is an endomorphin-1 peptide or an endomorphin-2 peptide. In another aspect of this embodiment, an endorphin peptide targeting domain an endorphin-a peptide, a neoendorphin-α peptide, an endorphin-β peptide, a neoendorphin-β peptide, or an endorphin-γ peptide. In another aspect of this embodiment, a dynorphin peptide targeting domain is a dynorphin A peptide, a dynorphin B (leumorphin) peptide, or a rimorphin peptide. In another aspect of this embodiment, a nociceptin peptide targeting domain is a nociceptin RK peptide, a nociceptin peptide, a neuropeptide 1 peptide, a neuropeptide 2 peptide, or a neuropeptide 3 peptide. In another aspect of this embodiment, a hemorphin peptide targeting domain is a LVVH7 peptide, a VVH7 peptide, a VH7 peptide, a H7 peptide, a LVVH6 peptide, a LVVH5 peptide, a VVH5 peptide, a LVVH4 peptide, or a LVVH3 peptide.

In an aspect of this embodiment, a galanin peptide targeting domain is a galanin peptide, a galanin message-associated peptide (GMAP) peptide, a galanin like protein (GALP) peptide, or an alarin peptide.

In an aspect of this embodiment, a PAR peptide targeting domain is a PAR1 peptide, a PAR2 peptide, a PAR3 peptide and a PAR4 peptide. In an aspect of this embodiment, a somatostatin peptide targeting domain is a somatostatin peptide or a cortistatin peptide. In an aspect of this embodiment, a neurotensin peptide targeting domain a neurotensin or a neuromedin N. In an aspect of this embodiment, a SLURP peptide targeting domain is a SLURP-1 peptide or a SLURP-2 peptide. In an aspect of this embodiment, an angiotensin peptide targeting domain is an angiotensin peptide.

In an aspect of this embodiment, a tachykinin peptide targeting domain is a Substance P peptide, a neuropeptide K peptide, a neuropeptide gamma peptide, a neurokinin A peptide, a neurokinin B peptide, a hemokinin peptide, or a endokinin peptide. In an aspect of this embodiment, a Neuropeptide Y related peptide targeting domain is a Neuropeptide Y peptide, a Peptide YY peptide, Pancreatic peptide peptide, a Pancreatic icosapeptide peptide, a Pancreatic Hormone domain peptide, a CXCL12 peptide, and a Sjogren syndrome antigen B peptide. In an aspect of this embodiment, a kinin peptide targeting domain is a bradykinin peptide, a kallidin peptide, a desArg9 bradykinin peptide, a desArg10 bradykinin peptide, a kininogen peptide, gonadotropin releasing hormone 1 peptide, chemokine peptide, an arginine vasopressin peptide.

In an aspect of this embodiment, a melanocortin peptide targeting domain comprises a melanocyte stimulating hormone peptide, an adrenocorticotropin peptide, a lipotropin peptide, or a melanocortin peptide derived neuropeptide. In an aspect of this embodiment, a melanocyte stimulating hormone peptide targeting domain comprises an α-melanocyte stimulating hormone peptide, a β-melanocyte stimulating hormone peptide, or a γ-melanocyte stimulating hormone peptide. In an aspect of this embodiment, an adrenocorticotropin peptide targeting domain comprises an adrenocorticotropin or a Corticotropin-like intermediary peptide. In an aspect of this embodiment, a lipotropin peptide targeting domain comprises a β-lipotropin peptide or a γ-lipotropin peptide.

In an aspect of this embodiment, a granin peptide targeting domain comprises a chromogranin A peptide, a chromogranin B peptide, a chromogranin C (secretogranin II) peptide, a secretogranin IV peptide, or a secretogranin VI peptide. In an aspect of this embodiment, a chromogranin A peptide targeting domain comprises a β-granin peptide, a vasostatin peptide, a chromostatin peptide, a pancreastatin peptide, a WE-14 peptide, a catestatin peptide, a parastatin peptide, or a GE-25 peptide. In an aspect of this embodiment, a chromogranin B peptide targeting domain comprises a GAWK peptide, an adrenomedullary peptide, or a secretolytin peptide. In an aspect of this embodiment, a chromogranin C peptide targeting domain comprises a secretoneurin peptide.

In an aspect of this embodiment, a glucagons-like hormone peptide targeting domain is a glucagon-like peptide-1, a glucagon-like peptide-2, a glicentin, a glicentin-related peptide (GRPP), a glucagon, or an oxyntomodulin (OXY). In an aspect of this embodiment, a secretin peptide targeting domain is a secretin peptide. In an aspect of this embodiment, a pituitary adenylate cyclase activating peptide targeting domain is a pituitary adenylate cyclase activating peptide. In an aspect of this embodiment, a growth hormone-releasing hormone peptide targeting domain a growth hormone-releasing hormone peptide. In an aspect of this embodiment, a vasoactive intestinal peptide targeting domain is a vasoactive intestinal peptide-1 peptide or a vasoactive intestinal peptide-2 peptide. In an aspect of this embodiment, a gastric inhibitory peptide targeting domain is a gastric inhibitory peptide. In an aspect of this embodiment, a calcitonin peptide targeting domain is a calcitonin peptide, an amylin peptide, a calcitonin-related peptide α, a calcitonin-related peptide β, and a islet amyloid peptide. In an aspect of this embodiment, a visceral gut peptide targeting domain is a gastrin peptide, a gastrin-releasing peptide, or a cholecystokinin peptide.

In an aspect of this embodiment, a neurotrophin peptide targeting domain is a nerve growth factor (NGF) peptide, a brain derived neurotrophic factor (BDNF) peptide, a neurotrophin-3 (NT-3) peptide, a neurotrophin-4/5 (NT-4/5) peptide, or an amyloid beta (A4) precursor protein neurotrophin (APP) peptide. In an aspect of this embodiment, a head activator peptide targeting domain is a head activator peptide. In an aspect of this embodiment, a glial cell line-derived neurotrophic factor family of ligands peptide targeting domain is a glial cell line-derived neurotrophic factor peptide, a Neurturin peptide, a Persephrin peptide, or an Artemin peptide. In an aspect of this embodiment, a RF-amide related peptide targeting domain a RF-amide related peptide-1, a RF-amide related peptide-2, a RF-amide related peptide-3, a neuropeptide AF, or a neuropeptide FF.

In an aspect of this embodiment, a neurohormone peptide targeting domain is a corticotropin-releasing hormone (CCRH), a parathyroid hormone (PTH), a parathyroid hormone-like hormone (PTHLH), a PHYH, a thyrotropin-releasing hormone (TRH), an urocortin-1 (UCN1), an urocortin-2 (UCN2), an urocortin-3 (UCN3), or an urotensin 2 (UTS2). In an aspect of this embodiment, a neuroregulatory cytokine peptide targeting domain is a ciliary neurotrophic factor peptide, a glycophorin-A peptide, a leukemia inhibitory factor peptide, a cardiotrophin-1 peptide, a cardiotrophin-like cytokine peptide, a neuroleukin peptide, and an onostatin M peptide. In an aspect of this embodiment, an IL peptide targeting domain is an IL-1 peptide, an IL-2 peptide, an IL-3 peptide, an IL-4 peptide, an IL-5 peptide, an IL-6 peptide, an IL-7 peptide, an IL-8 peptide, an IL-9 peptide, an IL-10 peptide, an IL-11 peptide, an IL-12 peptide, an IL-18 peptide, an IL-32 peptide, or an IL-33 peptide.

In an aspect of this embodiment, a VEGF peptide targeting domain is a VEGF-A peptide, a VEGF-B peptide, a VEGF-C peptide, a VEGF-D peptide, or a placenta growth factor (PIGF) peptide. In an aspect of this embodiment, an IGF peptide targeting domain is an IGF-1 peptide or an IGF-2 peptide. In an aspect of this embodiment, an EGF peptide targeting domain an EGF, a heparin-binding EGF-like growth factor (HB-EGF), a transforming growth factor-α (TGF-α), an amphiregulin (AR), an epiregulin (EPR), an epigen (EPG), a betacellulin (BTC), a neuregulin-1 (NRG1), a neuregulin-2 (NRG2), a neuregulin-3, (NRG3), or a neuregulin-4 (NRG4). In an aspect of this embodiment, a FGF peptide targeting domain is a FGF1 peptide, a FGF2 peptide, a FGF3 peptide, a FGF4 peptide, a FGF5 peptide, a FGF6 peptide, a FGF7 peptide, a FGF8 peptide, a FGF9 peptide, a FGF10 peptide, a FGF17 peptide, or a FGF18 peptide. In an aspect of this embodiment, a PDGF peptide targeting domain is a PDGFα peptide or a PDGFβ peptide.

In an aspect of this embodiment, a TGFβ peptide targeting domain is a TGFβ1 peptide, a TGFβ2 peptide, a TGFβ3 peptide, or a TGFβ4 peptide. In an aspect of this embodiment, a BMP peptide targeting domain is a BMP2 peptide, a BMP3 peptide, a BMP4 peptide, a BMP5 peptide, a BMP6 peptide, a BMP7 peptide, a BMP8 peptide, or a BMP10 peptide. In an aspect of this embodiment, a GDF peptide targeting domain is a GDF1 peptide, a GDF2 peptide, a GDF3 peptide, a GDF5 peptide, a GDF6 peptide, a GDF7 peptide, a GDF8 peptide, a GDF10 peptide, a GDF11 peptide, or a GDF15 peptide. In an aspect of this embodiment, an activin peptide targeting domain is an activin A peptide, an activin B peptide, an activin C peptide, an activin E peptide, or an inhibin A peptide.

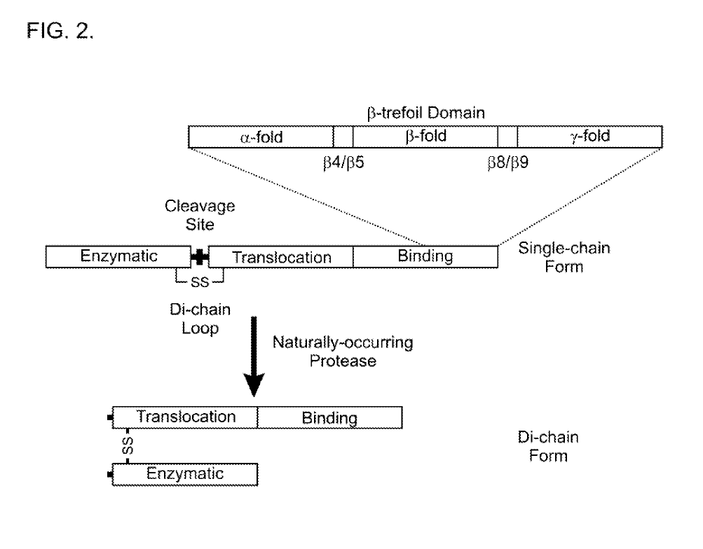

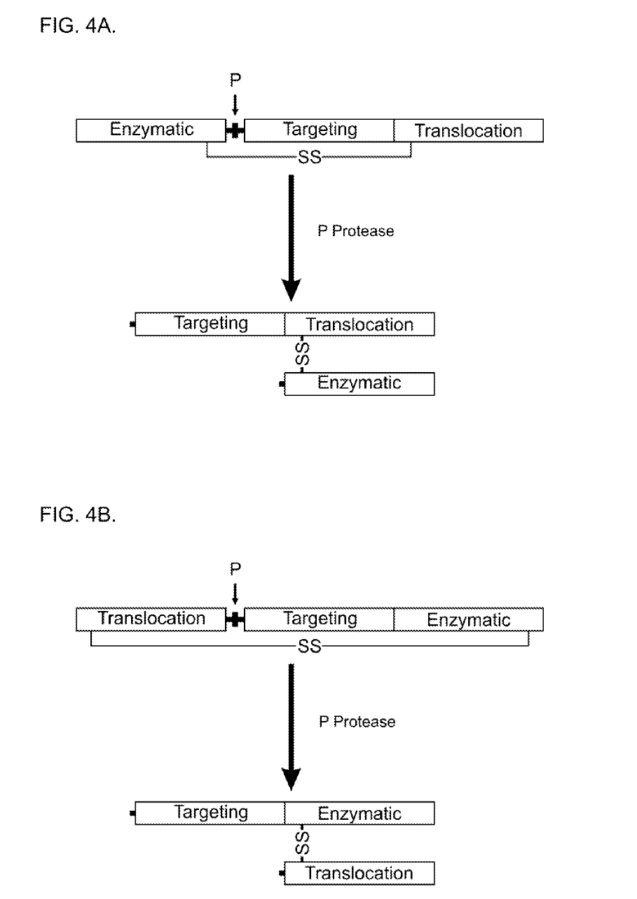

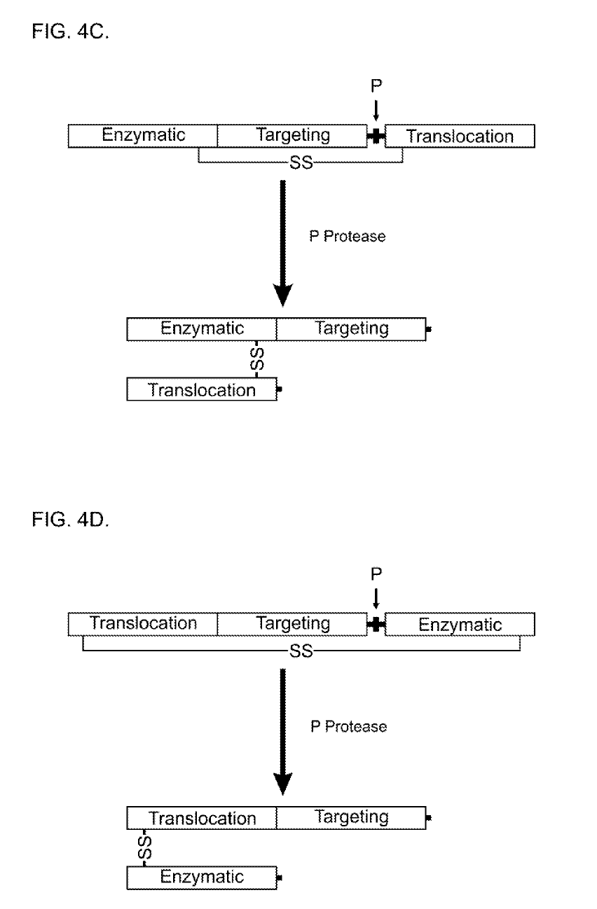

As discussed above, naturally-occurring Clostridial toxins are organized into three functional domains comprising a linear amino-to-carboxyl single polypeptide order of the enzymatic domain (amino region position), the translocation domain (middle region position) and the binding domain (carboxyl region position) (FIG. 2). This naturally-occurring order can be referred to as the carboxyl presentation of the binding domain because the domain necessary for binding to the receptor is located at the carboxyl region position of the Clostridial toxin. However, it has been shown that Clostridial toxins can be modified by rearranging the linear amino-to-carboxyl single polypeptide order of the three major domains and locating a targeting moiety at the amino region position of a Clostridial toxin, referred to as amino presentation, as well as in the middle region position, referred to as central presentation (FIG. 4).

Thus, a TEM can comprise a targeting domain in any and all locations with the proviso that TEM is capable of performing the intoxication process. Non-limiting examples include, locating a targeting domain at the amino terminus of a TEM; locating a targeting domain between a Clostridial toxin enzymatic domain and a Clostridial toxin translocation domain of a TEM; and locating a targeting domain at the carboxyl terminus of a TEM. Other non-limiting examples include, locating a targeting domain between a Clostridial toxin enzymatic domain and a Clostridial toxin translocation domain of a TEM. The enzymatic domain of naturally-occurring Clostridial toxins contains the native start methionine. Thus, in domain organizations where the enzymatic domain is not in the amino-terminal location an amino acid sequence comprising the start methionine should be placed in front of the amino-terminal domain. Likewise, where a targeting domain is in the amino-terminal position, an amino acid sequence comprising a start methionine and a protease cleavage site may be operably-linked in situations in which a targeting domain requires a free amino terminus, see, e.g., Shengwen Li et al., Degradable Clostridial Toxins, U.S. patent application Ser. No. 11/572,512 (Jan. 23, 2007), which is hereby incorporated by reference in its entirety. In addition, it is known in the art that when adding a polypeptide that is operably-linked to the amino terminus of another polypeptide comprising the start methionine that the original methionine residue can be deleted.

A TEM disclosed herein may optionally comprise an exogenous protease cleavage site that allows the use of an exogenous protease to convert the single-chain polypeptide form of a TEM into its more active di-chain form. As used herein, the term “exogenous protease cleavage site” is synonymous with a “non-naturally occurring protease cleavage site” or “non-native protease cleavage site” and means a protease cleavage site that is not naturally found in a di-chain loop region from a naturally occurring Clostridial toxin.

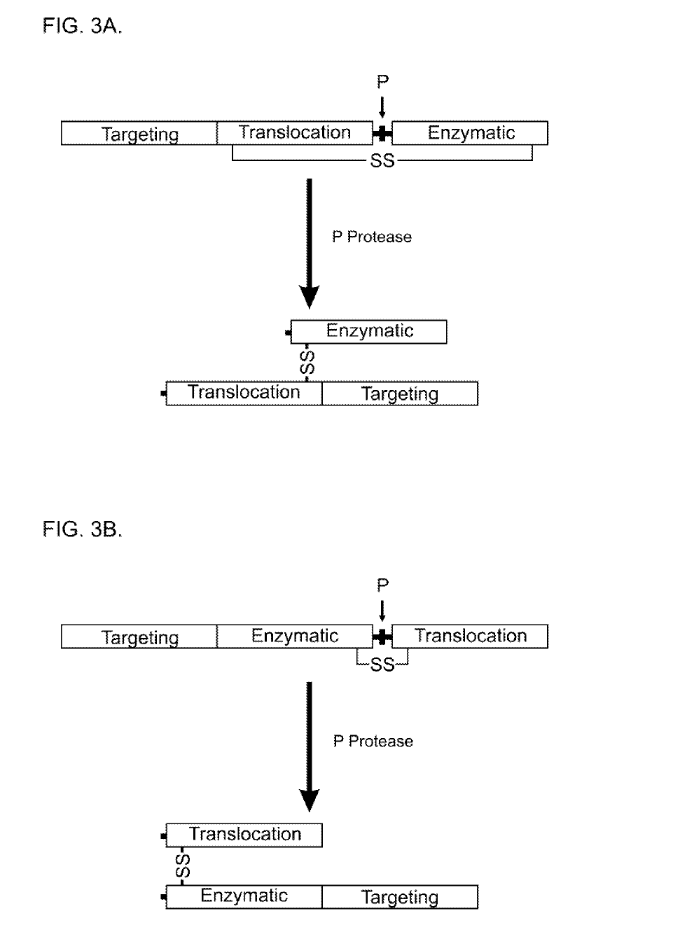

Naturally-occurring Clostridial toxins are each translated as a single-chain polypeptide of approximately 150 kDa that is subsequently cleaved by proteolytic scission within a disulfide loop by a naturally-occurring protease (FIG. 2). This cleavage occurs within the discrete di-chain loop region located between two cysteine residues that form a disulfide bridge and comprising an endogenous protease cleavage site. As used herein, the term “endogenous di-chain loop protease cleavage site” is synonymous with a “naturally occurring di-chain loop protease cleavage site” and refers to a naturally occurring protease cleavage site found within the di-chain loop region of a naturally occurring Clostridial toxin. This posttranslational processing yields a di-chain molecule comprising an approximately 50 kDa light chain, comprising the enzymatic domain, and an approximately 100 kDa heavy chain, comprising the translocation and cell binding domains, the light chain and heavy chain being held together by the single disulfide bond and non-covalent interactions (FIG. 2). Recombinantly-produced Clostridial toxins generally substitute the naturally-occurring di-chain loop protease cleavage site with an exogenous protease cleavage site to facilitate production of a recombinant di-chain molecule (FIGS. 3-5). See e.g., Dolly, J. O. et al., Activatable Clostridial Toxins, U.S. Pat. No. 7,419,676 (Sep. 2, 2008), which is hereby incorporated by reference.

Although TEMs vary in their overall molecular weight because the size of the targeting domain, the activation process and its reliance on an exogenous cleavage site is essentially the same as that for recombinantly-produced Clostridial toxins. See e.g., Steward, et al., Activatable Clostridial Toxins, US 2009/0081730; Steward, et al., Modified Clostridial Toxins with Enhanced Translocation Capabilities and Altered Targeting Activity For Non-Clostridial Toxin Target Cells, U.S. patent application Ser. No. 11/776,075; Steward, et al., Modified Clostridial Toxins with Enhanced Translocation Capabilities and Altered Targeting Activity for Clostridial Toxin Target Cells, US 2008/0241881, each of which is hereby incorporated by reference. In general, the activation process that converts the single-chain polypeptide into its di-chain form using exogenous proteases can be used to process TEMs having a targeting domain organized in an amino presentation, central presentation, or carboxyl presentation arrangement. This is because for most targeting domains the amino-terminus of the moiety does not participate in receptor binding. As such, a wide range of protease cleavage sites can be used to produce an active di-chain form of a TEM. However, targeting domains requiring a free amino-terminus for receptor binding require a protease cleavage site whose scissile bond is located at the carboxyl terminus. The use of protease cleavage site is the design of a TEM are described in, e.g., Steward, et al., Activatable Clostridial toxins, US 2009/0069238; Ghanshani, et al., Modified Clostridial Toxins Comprising an Integrated Protease Cleavage Site-Binding Domain, US 2011/0189162; and Ghanshani, et al., Methods of Intracellular Conversion of Single-Chain Proteins into their Di-chain Form, International Patent Application Serial No. PCT/US2011/22272, each of which is incorporated by reference in its entirety.

Non-limiting examples of exogenous protease cleavage sites include, e.g., a plant papain cleavage site, an insect papain cleavage site, a crustacian papain cleavage site, an enterokinase protease cleavage site, a Tobacco Etch Virus protease cleavage site, a Tobacco Vein Mottling Virus protease cleavage site, a human rhinovirus 3C protease cleavage site, a human enterovirus 3C protease cleavage site, a subtilisin cleavage site, a hydroxylamine cleavage site, a SUMO/ULP-1 protease cleavage site, and a Caspase 3 cleavage site.

Thus, in an embodiment, a TEM can comprise an amino to carboxyl single polypeptide linear order comprising a targeting domain, a translocation domain, an exogenous protease cleavage site and an enzymatic domain (FIG. 3A). In an aspect of this embodiment, a TEM can comprise an amino to carboxyl single polypeptide linear order comprising a targeting domain, a Clostridial toxin translocation domain, an exogenous protease cleavage site and a Clostridial toxin enzymatic domain.

In another embodiment, a TEM can comprise an amino to carboxyl single polypeptide linear order comprising a targeting domain, an enzymatic domain, an exogenous protease cleavage site, and a translocation domain (FIG. 3B). In an aspect of this embodiment, a TEM can comprise an amino to carboxyl single polypeptide linear order comprising a targeting domain, a Clostridial toxin enzymatic domain, an exogenous protease cleavage site, a Clostridial toxin translocation domain.

In yet another embodiment, a TEM can comprise an amino to carboxyl single polypeptide linear order comprising an enzymatic domain, an exogenous protease cleavage site, a targeting domain, and a translocation domain (FIG. 4A). In an aspect of this embodiment, a TEM can comprise an amino to carboxyl single polypeptide linear order comprising a Clostridial toxin enzymatic domain, an exogenous protease cleavage site, a targeting domain, and a Clostridial toxin translocation domain.

In yet another embodiment, a TEM can comprise an amino to carboxyl single polypeptide linear order comprising a translocation domain, an exogenous protease cleavage site, a targeting domain, and an enzymatic domain (FIG. 4B). In an aspect of this embodiment, a TEM can comprise an amino to carboxyl single polypeptide linear order comprising a Clostridial toxin translocation domain, a targeting domain, an exogenous protease cleavage site and a Clostridial toxin enzymatic domain.

In another embodiment, a TEM can comprise an amino to carboxyl single polypeptide linear order comprising an enzymatic domain, a targeting domain, an exogenous protease cleavage site, and a translocation domain (FIG. 4C). In an aspect of this embodiment, a TEM can comprise an amino to carboxyl single polypeptide linear order comprising a Clostridial toxin enzymatic domain, a targeting domain, an exogenous protease cleavage site, a Clostridial toxin translocation domain.

In yet another embodiment, a TEM can comprise an amino to carboxyl single polypeptide linear order comprising a translocation domain, a targeting domain, an exogenous protease cleavage site and an enzymatic domain (FIG. 4D). In an aspect of this embodiment, a TEM can comprise an amino to carboxyl single polypeptide linear order comprising a Clostridial toxin translocation domain, a targeting domain, an exogenous protease cleavage site and a Clostridial toxin enzymatic domain.

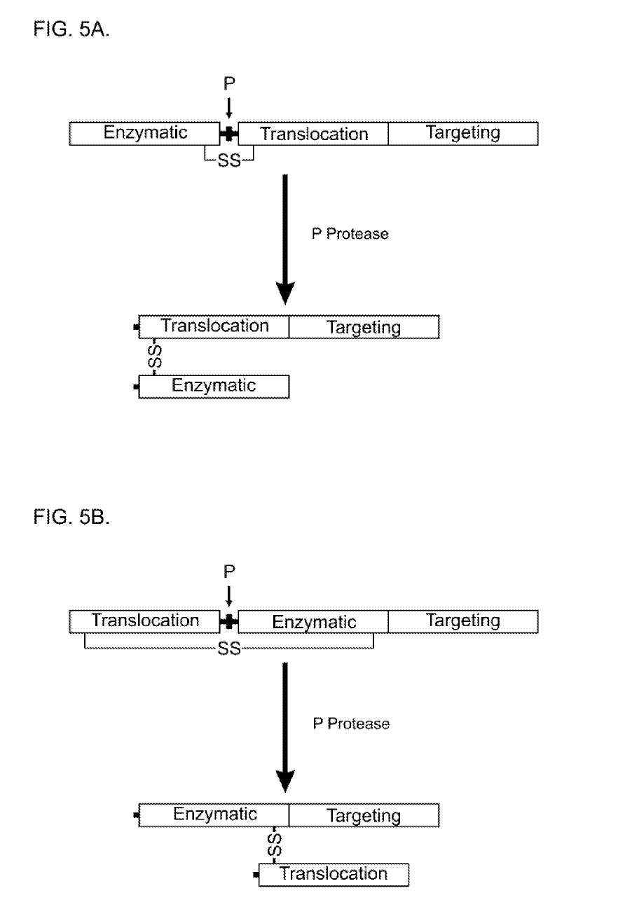

In still another embodiment, a TEM can comprise an amino to carboxyl single polypeptide linear order comprising an enzymatic domain, an exogenous protease cleavage site, a translocation domain, and a targeting domain (FIG. 5A). In an aspect of this embodiment, a TEM can comprise an amino to carboxyl single polypeptide linear order comprising a Clostridial toxin enzymatic domain, an exogenous protease cleavage site, a Clostridial toxin translocation domain, and a targeting domain.

In still another embodiment, a TEM can comprise an amino to carboxyl single polypeptide linear order comprising a translocation domain, an exogenous protease cleavage site, an enzymatic domain and a targeting domain, (FIG. 5B). In an aspect of this embodiment, a TEM can comprise an amino to carboxyl single polypeptide linear order comprising a Clostridial toxin translocation domain, a targeting domain, an exogenous protease cleavage site and a Clostridial toxin enzymatic domain.

Non-limiting examples of TEMs disclosed herein, including TEMs comprising a Clostridal toxin enzymatic domain, a Clostridial toxin translocation domain and a targeting domain, the use of an exogenous protease cleavage site, and the design of amino presentation, central presentation and carboxyl presentation TEMs are described in, e.g., U.S. Pat. No. 7,959,933, Activatable Recombinant Neurotoxins, U.S. Pat. No. 7,897,157, Activatable Clostridial Toxins; U.S. Pat. No. 7,833,535, Clostridial Toxin Derivatives and Methods for Treating Pain; U.S. Pat. No. 7,811,584, Multivalent Clostridial Toxins; U.S. Pat. No. 7,780,968, Clostridial Toxin Derivatives and Methods for Treating Pain; U.S. Pat. No. 7,749,514, Activatable Clostridial Toxins, U.S. Pat. No. 7,740,868, Activatable Clostridial Toxins; U.S. Pat. No. 7,736,659, Clostridial Toxin Derivatives and Methods for Treating Pain; U.S. Pat. No. 7,709,228, Activatable Recombinant Neurotoxins; U.S. Pat. No. 7,704,512, Clostridial Toxin Derivatives and Methods for Treating Pain; U.S. Pat. No. 7,659,092, Fusion Proteins; U.S. Pat. No. 7,658,933, Non-Cytotoxic Protein Conjugates; U.S. Pat. No. 7,622,127, Clostridial Toxin Derivatives and Methods for Treating Pain; U.S. Pat. No. 7,514,088, Multivalent Clostridial Toxin Derivatives and Methods of Their Use; U.S. Pat. No. 7,425,338, Clostridial Toxin Derivatives and Methods for Treating Pain; U.S. Pat. No. 7,422,877, Activatable Recombinant Neurotoxins; U.S. Pat. No. 7,419,676, Activatable Recombinant Neurotoxins; U.S. Pat. No. 7,413,742, Clostridial Toxin Derivatives and Methods for Treating Pain; U.S. Pat. No. 7,262,291, Clostridial Toxin Derivatives and Methods for Treating Pain; U.S. Pat. No. 7,244,437, Clostridial Toxin Derivatives and Methods for Treating Pain; U.S. Pat. No. 7,244,436, Clostridial Toxin Derivatives and Methods for Treating Pain; U.S. Pat. No. 7,138,127, Clostridial Toxin Derivatives and Methods for Treating Pain; U.S. Pat. No. 7,132,259, Activatable Recombinant Neurotoxins; U.S. Pat. No. 7,056,729, Botulinum Neurotoxin-Substance P Conjugate or Fusion Protein for Treating Pain; U.S. Pat. No. 6,641,820, Clostridial Toxin Derivatives and Methods to Treat Pain; U.S. Pat. No. 6,500,436, Clostridial Toxin Derivatives and Methods for Treating Pain; US 2011/0091437, Fusion Proteins; US 2011/0070621, Multivalent Clostridial Toxins; US 2011/0027256, Fusion Proteins; US 2010/0247509, Fusion Proteins; US 2010/0041098, Modified Clostridial Toxins with Altered Targeting Capabilities for Clostridial Toxin Target Cells; US 2010/0034802, Treatment of Pain; US 2009/0162341, Non-Cytotoxic Protein Conjugates; US 2009/0087458, Activatable Recombinant Neurotoxins; US 2009/0081730, Activatable Recombinant Neurotoxins; US 2009/0069238, Activatable Clostridial Toxins; US 2009/0042270, Activatable Recombinant Neurotoxins; US 2009/0030182, Activatable Recombinant Neurotoxins; US 2009/0018081, Activatable Clostridial Toxins; US 2009/0005313, Activatable Clostridial Toxins; US 2009/0004224, Activatable Clostridial Toxins; US 2008/0317783, Clostridial Toxin Derivatives and Methods for Treating Pain; US 2008/0241881, Modified Clostridial Toxins with Enhanced Translocation Capabilities and Altered Targeting Activity for Clostridial Toxin Target Cells; WO 2006/099590, Modified Clostridial Toxins with Altered Targeting Capabilities for Clostridial Toxin Target Cells; WO 2006/101809, Modified Clostridial Toxins with Enhanced Targeting Capabilities for Endogenous Clostridial Toxin Receptor Systems; WO 2007/106115, Modified Clostridial Toxins with Altered Targeting Capabilities for Clostridial Toxin Target Cells; WO 2008/008803, Modified Clostridial Toxins with Enhanced Translocation Capabilities and Altered Targeting Activity for Clostridial Toxin Target Cells; WO 2008/008805, Modified Clostridial Toxins with Enhanced Translocation Capabilities and Altered Targeting Activity For Non-Clostridial Toxin Target Cells; WO 2008/105901, Modified Clostridial Toxins with Enhanced Translocation Capability and Enhanced Targeting Activity; WO 2011/020052, Methods of Treating Cancer Using Opioid Retargeted Endpeptidases; WO 2011/020056, Methods of Treating Cancer Using Galanin Retargeted Endpeptidases; WO 2011/020114, Methods of Treating Cancer Using Tachykinin Retargeted Endopeptidases; WO 2011/020115, Methods of Treating Cancer Using Growth Factor Retargeted Endopeptidases; WO 2011/020117, Methods of Treating Cancer Using Neurotrophin Retargeted Endopeptidases; WO 2011/020119, Methods of Treating Cancer Using Glucagon-Like Hormone Retargeted Endopeptidases; each of which is incorporated by reference in its entirety.

A composition disclosed herein is generally administered as a pharmaceutical acceptable composition. As used herein, the term “pharmaceutically acceptable” means any molecular entity or composition that does not produce an adverse, allergic or other untoward or unwanted reaction when administered to an individual. As used herein, the term “pharmaceutically acceptable composition” is synonymous with “pharmaceutical composition” and means a therapeutically effective concentration of an active ingredient, such as, e.g., any of the Clostridial toxins and/or TEMs disclosed herein. A pharmaceutical composition disclosed herein is useful for medical and veterinary applications. A pharmaceutical composition may be administered to an individual alone, or in combination with other supplementary active ingredients, agents, drugs or hormones. The pharmaceutical compositions may be manufactured using any of a variety of processes, including, without limitation, conventional mixing, dissolving, granulating, dragee-making, levigating, emulsifying, encapsulating, entrapping, and lyophilizing. The pharmaceutical composition can take any of a variety of forms including, without limitation, a sterile solution, suspension, emulsion, lyophilizate, tablet, pill, pellet, capsule, powder, syrup, elixir or any other dosage form suitable for administration.

A pharmaceutical composition disclosed herein may optionally include a pharmaceutically acceptable carrier that facilitates processing of an active ingredient into pharmaceutically acceptable compositions. As used herein, the term “pharmacologically acceptable carrier” is synonymous with “pharmacological carrier” and means any carrier that has substantially no long term or permanent detrimental effect when administered and encompasses terms such as “pharmacologically acceptable vehicle, stabilizer, diluent, additive, auxiliary or excipient.” Such a carrier generally is mixed with an active ingredient, or permitted to dilute or enclose the active compound and can be a solid, semi-solid, or liquid agent. It is understood that the active ingredients can be soluble or can be delivered as a suspension in the desired carrier or diluent. Any of a variety of pharmaceutically acceptable carriers can be used including, without limitation, aqueous media such as, e.g., water, saline, glycine, hyaluronic acid and the like; solid carriers such as, e.g., mannitol, lactose, starch, magnesium stearate, sodium saccharin, talcum, cellulose, glucose, sucrose, magnesium carbonate, and the like; solvents; dispersion media; coatings; antibacterial and antifungal agents; isotonic and absorption delaying agents; or any other inactive ingredient. Selection of a pharmacologically acceptable carrier can depend on the mode of administration. Except insofar as any pharmacologically acceptable carrier is incompatible with the active ingredient, its use in pharmaceutically acceptable compositions is contemplated. Non-limiting examples of specific uses of such pharmaceutical carriers can be found in PHARMACEUTICAL DOSAGE FORMS AND DRUG DELIVERY SYSTEMS (Howard C. Ansel et al., eds., Lippincott Williams & Wilkins Publishers, 7^{th} ed. 1999); REMINGTON: THE SCIENCE AND PRACTICE OF PHARMACY (Alfonso R. Gennaro ed., Lippincott, Williams & Wilkins, 20^{th} ed. 2000); GOODMAN & GILMAN'S THE PHARMACOLOGICAL BASIS OF THERAPEUTICS (Joel G. Hardman et al., eds., McGraw-Hill Professional, 10^{th} ed. 2001); and HANDBOOK OF PHARMACEUTICAL EXCIPIENTS (Raymond C. Rowe et al., APhA Publications, 4^{th} edition 2003). These protocols are routine procedures and any modifications are well within the scope of one skilled in the art and from the teaching herein.

A pharmaceutical composition disclosed herein can optionally include, without limitation, other pharmaceutically acceptable components (or pharmaceutical components), including, without limitation, buffers, preservatives, tonicity adjusters, salts, antioxidants, osmolality adjusting agents, physiological substances, pharmacological substances, bulking agents, emulsifying agents, wetting agents, sweetening or flavoring agents, and the like. Various buffers and means for adjusting pH can be used to prepare a pharmaceutical composition disclosed herein, provided that the resulting preparation is pharmaceutically acceptable. Such buffers include, without limitation, acetate buffers, citrate buffers, phosphate buffers, neutral buffered saline, phosphate buffered saline and borate buffers. It is understood that acids or bases can be used to adjust the pH of a composition as needed. Pharmaceutically acceptable antioxidants include, without limitation, sodium metabisulfite, sodium thiosulfate, acetylcysteine, butylated hydroxyanisole and butylated hydroxytoluene. Useful preservatives include, without limitation, benzalkonium chloride, chlorobutanol, thimerosal, phenylmercuric acetate, phenylmercuric nitrate, a stabilized oxy chloro composition and chelants, such as, e.g., DTPA or DTPA-bisamide, calcium DTPA, and CaNaDTPA-bisamide. Tonicity adjustors useful in a pharmaceutical composition include, without limitation, salts such as, e.g., sodium chloride, potassium chloride, mannitol or glycerin and other pharmaceutically acceptable tonicity adjustor. The pharmaceutical composition may be provided as a salt and can be formed with many acids, including but not limited to, hydrochloric, sulfuric, acetic, lactic, tartaric, malic, succinic, etc. Salts tend to be more soluble in aqueous or other protonic solvents than are the corresponding free base forms. It is understood that these and other substances known in the art of pharmacology can be included in a pharmaceutical composition. Exemplary pharmaceutical composition comprising a TEM are described in Hunt, et al., Animal Protein-Free Pharmaceutical Compositions, U.S. Ser. No. 12/331,816; and Dasari, et al., Clostridial Toxin Pharmaceutical Compositions, WO/2010/090677, each of which is hereby incorporated by reference in its entirety.

In an embodiment, a composition is a pharmaceutical composition comprising a TEM. In aspects of this embodiment, a pharmaceutical composition comprising a TEM further comprises a pharmacological carrier, a pharmaceutical component, or both a pharmacological carrier and a pharmaceutical component. In other aspects of this embodiment, a pharmaceutical composition comprising a TEM further comprises at least one pharmacological carrier, at least one pharmaceutical component, or at least one pharmacological carrier and at least one pharmaceutical component.

In another embodiment, a composition is a pharmaceutical composition comprising a Clostridial toxin. In aspects of this embodiment, a pharmaceutical composition comprising a Clostridial toxin further comprises a pharmacological carrier, a pharmaceutical component, or both a pharmacological carrier and a pharmaceutical component. In other aspects of this embodiment, a pharmaceutical composition comprising a Clostridial toxin further comprises at least one pharmacological carrier, at least one pharmaceutical component, or at least one pharmacological carrier and at least one pharmaceutical component.

In yet another embodiment, a composition is a pharmaceutical composition comprising a Clostridial toxin and a TEM. In aspects of this embodiment, a pharmaceutical composition comprising a Clostridial toxin and a TEM further comprises a pharmacological carrier, a pharmaceutical component, or both a pharmacological carrier and a pharmaceutical component. In other aspects of this embodiment, a pharmaceutical composition comprising a Clostridial toxin and a TEM further comprises at least one pharmacological carrier, at least one pharmaceutical component, or at least one pharmacological carrier and at least one pharmaceutical component.

Aspects of the present specification disclose, in part, treating an individual suffering from a sexual dysfunction disorder. As used herein, the term “treating,” refers to reducing or eliminating in an individual a clinical symptom of a sexual dysfunction disorder; or delaying or preventing in an individual the onset of a clinical symptom of a sexual dysfunction disorder. For example, the term “treating” can mean reducing a symptom of a condition characterized by a sexual dysfunction disorder by, e.g., at least 20%, at least 30%, at least 40%, at least 50%, at least 60%, at least 70%, at least 80%, at least 90% or at least 100%. The actual symptoms associated with a sexual dysfunction disorder are well known and can be determined by a person of ordinary skill in the art by taking into account factors, including, without limitation, the location of the sexual dysfunction disorder, the cause of the sexual dysfunction disorder, the severity of the sexual dysfunction disorder, and/or the tissue or organ affected by the sexual dysfunction disorder. Those of skill in the art will know the appropriate symptoms or indicators associated with specific sexual dysfunction disorder and will know how to determine if an individual is a candidate for treatment as disclosed herein.

As used herein, the term “sexual dysfunction disorder” refers to a sexual dysfunction disorder where at least one of the underlying symptoms being treated is due to a sensory nerve-based etiology, a sympathetic nerve-based etiology, and/or a parasympathetic nerve-based etiology. Typically such etiologies will involve an abnormal overactivity of a nerve that results in symptoms of a sexual dysfunction disorder, or any normal activity of a nerve that needs to be reduced or stopped for a period of time in order to treat a sexual dysfunction disorder. A sexual dysfunction disorder can be a condition that makes difficult, reduces, or prevents an individual's enjoyment of normal sexual activity, including, e.g., desire, arousal, or orgasm; and/or makes difficult, reduces, or prevents the normal physiological changes brought on normally by such activity. Sexual dysfunction disorders include, without limitation, a sexual desire disorder, a sexual arousal disorder, a sexual orgasm disorder, a sexual pain disorder, a sexsomnia, and a climacturia.

A sexual desire disorder refers to a sexual dysfunction disorder where an individual has an abnormal desire or libido for sexual thoughts or fantasies and/or desire for sexual activity and includes both a complete aversion to sexual thoughts, fantasies, or activities and a complete preoccupation with sexual thoughts, fantasies, or activities. A sexual desire disorder includes situations where there is a significant difference in desire or libido between an individual and his/her partner or where an individual alone has an abnormal distaste for or fixation with sexual thoughts, fantasies, or activities. Sexual desire disorders include, without limitation, a hypoactive sexual desire disorder, a sexual aversion disorder, and a hyperactive sexual desire disorder.

A hypoactive sexual desire disorder (also known as inhibited sexual desire disorder) refers to a condition where sexual thoughts or fantasies and/or desire for sexual activity is persistently reduced or absent in an individual. The condition ranges from a reduced or complete lack of sexual desire to engage in any type of sexual thought, fantasy, or activity with the current partner to a reduced or complete lack of sexual desire to engage in any type of sexual thought, fantasy, or activity generally. The condition may have started after a period of normal sexual functioning or the individual may always have had no/low sexual desire. An individual with a hypoactive sexual desire disorder has little or no interest in engaging in any type of sexual activity, has few sexual thoughts or fantasies, and has a lack of sexual response.

A sexual aversion disorder refers to a condition where an individual is unusually apprehensive or repulsed by the thought of engaging in any type of sexual thoughts, fantasies or activity. The condition ranges from an unusually apprehension or repulsion to engage in any type of sexual thought, fantasy, or activity with the current partner to having such apprehension or repulsion generally. The condition may have started after a period of normal sexual functioning or the individual may always have had an aversion to sexual thoughts, fantasies, and/or activities. An individual with a sexual aversion disorder has little or no interest in engaging in any type of sexual activity, avoids genital sexual contact, has few or no sexual thoughts or fantasies, and has a lack of sexual response.

A hyperactive sexual desire disorder (also known as hypersexual desire disorder, sex addiction, sexual compulsivity, nymphomania or satyriasis) refers to a condition where sexual thoughts or fantasies and/or desire for sexual activity are abnormally persistently present in an individual. This disorder lies at the other end of the spectrum for hypoactive sexual desire disorder. An individual with a hyperactive sexual desire disorder typically experiences significant personal distress or impairment in social, occupational or other important areas of functioning. The condition ranges from having sexual thoughts or fantasies and/or the desire to engage in any type of sexual activity with the current partner to having such thoughts, fantasies and/or desires generally. The condition may have started after a period of normal sexual functioning or the individual may always have had a persistent or heightened sexual desire. An individual with a hyperactive sexual desire disorder is constantly interest in engaging in any type of sexual activity; is preoccupied with sexual thoughts or fantasies; has recurrent and intense sexual thoughts, urges or fantasies; repeatedly has sexual thoughts, fantasies, or urges in response to anxiety, depression, boredom, irritability, or stress; is preoccupied with planning or engaging in a sexual activity; is very demanding sexually; has a quick sexual response; abnormally engages in sexual activity; repeatedly engages in sexual activity in response to anxiety, depression, boredom, irritability, or stress; repeatedly being unsuccessful at trying to control sexual thoughts, fantasies, urges, or behavior; and repeatedly engaging in sexual activity despite physical or emotional risk to individual or others.

A sexual arousal disorder refers to a sexual dysfunction disorder where an individual has an abnormal arousal response to sexual stimulation including, but not limited to, physical, emotional, and/or mental stimulation. A sexual arousal disorder includes situations where there is an abnormal arousal response to sexual stimulation from a partner or from the individual. Sexual arousal disorders include, without limitation, a deficient sexual arousal disorder, a persistent sexual arousal disorder, and a priapism.

A deficient sexual arousal disorder refers to a condition where there is a lack of an arousal response in an individual after sexual stimulation. These disorders include situations where physical sexual stimulation fails to evoke a sexual response in an individual; or where even thought the individual has a desire for sexual activity and is physically sexually aroused, the individual does not feel sexually aroused.

A deficient sexual arousal disorder can be classified into several types, which can occur alone or in combination with other disorders. Subjective deficient sexual arousal disorders refer to conditions where regardless of the amount or degree of sexual stimulation the individual feels no or little mental sexual arousal. Thus, a female can exhibit increased vaginal lubrication or a male can exhibit a penile erection, but the person does not feel sexually aroused. Genital deficient sexual arousal disorders refer to conditions where regardless of the amount or degree of sexual stimulation the individual physically exhibits no or minimal arousal response even though the individual has the desire to engage in sexual activity. Thus, a female can exhibit no vaginal lubrication or swelling of the vulva while a male will not have a penile erection. Combined genital and subjective deficient arousal disorders refers to conditions where regardless of the amount or degree of sexual stimulation there is no or minimal physical manifestation of an arousal response and the individual has no or minimal mental sexual arousal.

Sexual arousal disorders were previously known as impotence in men and frigidity in women. Modernly, impotence is now known as erectile dysfunction and refers to the inability of a male to attain or sustain a penile erection long enough for coitus or other sexual activity. Erectile dysfunction can occur due to both physiological and psychological reasons. Common physiological reasons include diabetes, kidney disease, chronic alcoholism, multiple sclerosis, atherosclerosis, vascular disease, and neurologic disease which collectively account for about 70 percent of ED cases. Some drugs used to treat other conditions, such as lithium and paroxetine, may cause erectile dysfunction.

Frigidity is simply referred to as a sexual arousal disorder, but may be further characterized by terms describing specific problem. This disorder involves the persistent or recurrent inability to reach or sustain the lubrication and swelling reaction in the arousal phase of the sexual response to the point that it causes personal distress. It is the second most common sexual problem among women, affecting an estimated 20% of women, and most frequently occurs in postmenopausal women. Low estrogen levels after menopause can make vaginal tissue dry and thin and reduce blood flow to genitals. As a result, the arousal phase of the sexual response may take longer and sensitivity of the vaginal area may decline. However, this can happen at any age.

A persistent sexual arousal disorder (also known as persistent genital arousal disorder, persistent sexual arousal syndrome, restless genital syndrome, and mempin syndrome) refers to a condition where there is a spontaneous, persistent, and uncontrollable genital arousal in the absence of any sexual stimulation in an individual. This arousal is unrelated to any sexual thoughts or desires and can occur with or without genital engorgement and/or orgasm, and can last for hours or days. An individual with persistent sexual arousal disorder experiences sexual arousal lasting for an extended period of time and arousal does not go away on its own; sexual arousal not related to sexually desire or stimulation; sexual arousal triggered by nonsexual events or by nothing at all; persistent physical signs of sexual arousal after orgasm; dissipation of physical signs of sexual arousal only after multiple orgasms; intrusive and unwanted sexual arousal; distress.

A priapism (also known as hulseyism) refers to a condition where there is a persistent erection of the penis or clitoris in the absence of sexual stimulation that lasts for at least four hours. Major types of priapism include low-flow priapism and high-flow priapism. Low-flow priapism involves a situation where there is inadequate blood flow from the penis or clitoris back to the body which prevents the return of the organ to its flaccid state, thereby resulting in a persistent erection. High-flow priapism involves a situation where there is excessive blood flow into the penis or clitoris, thereby resulting in a persistent erection.

A sexual orgasm disorder refers to a sexual dysfunction disorder where an individual experiences an abnormal organism or ejaculation. Both men and women can have a variety of problems with orgasm. Sexual orgasmic disorders generally fall into one of several categories including where, during normal sexual activity, orgasms are absence (anorgasmia), orgasms are delayed or difficult to achieve, orgasms are too rapid, or orgasms of diminished orgasmic sensations.

In males, the common sexual orgasm disorders include male orgasmic disorder, male anorgasmia, premature ejaculation, and ejaculatory incompetence. Male orgasmic disorder refers to a condition where there is a delay in orgasm even after sufficient sexual stimulation and arousal. The disorder can have physical, psychological, or pharmacological origins.

Male anorgasmia refers to a condition where there is an absence in orgasm even after sufficient sexual stimulation and arousal. The disorder can have physical, psychological, or pharmacological origins.

Premature ejaculation refers to a condition where a male is unable to control ejaculation so that it occurs before satisfying sexual relations can take place with the partner, such as, e.g., a man ejaculates after engaging in sexual activity for only a very short period of time. Ejaculation and orgasm are two separate events in men, but because they usually happen simultaneously premature ejaculation is often considered an orgasmic disorder for men. Premature ejaculation is considered to be the most common male sexual complaint, and it can lead to a reduction in the pleasure of orgasm, given that it is accompanied by distress, feelings of shame or frustration. Premature ejaculation includes lifelong premature ejaculation, acquired premature ejaculation, natural variable premature ejaculation and premature-like ejaculatory dysfunction. The syndromes differ in pathophysiology, ejaculation time duration, frequency and pattern of premature ejaculation complaints, and its course in life. The underlying causes of premature ejaculation are still not understood.

Ejaculatory incompetence refers to a condition where a male experiences a delay or absence in reaching orgasm or ejaculation after sufficient sexual stimulation and arousal. The disorder can have physical, psychological, or pharmacological origins.

In females, common sexual orgasm disorders include female orgasmic disorder, female anorgasmia, and inhibited female orgasm. A female orgasmic disorder is a condition where there is a delay in orgasm even after sufficient sexual stimulation and arousal. The disorder can have physical, psychological, or pharmacological origins.

A female anorgasmia disorder refers to a condition where there is an absence in orgasm even after sufficient sexual stimulation and arousal. Female anorgasmia is further broken down into primary anorgasmia which refers to a woman never having had an orgasm, and secondary anorgasmia where a woman has previously experienced orgasms but isn't anymore, or where she can only experience orgasms under specific conditions, such as, e.g., only through masturbation but not during sexual intercourse. A female anorgasmia disorder may be temporary or persistent. The disorder can have physical, psychological, or pharmacological origins.

Inhibited female orgasm refers to a condition where there is a delay or absence in reaching orgasm after sufficient sexual stimulation and arousal. The disorder can have physical, psychological, or pharmacological origins.

A sexual pain disorder refers to a sexual dysfunction disorder where pain typically occurs during or after sex, and may happen in response to arousal, to stimulation, or to orgasm. Both men and women report sexual pain disorders, but they are reported much more among women. Sexual pain disorders include, without limitation, dyspareunia, vaginismus, vulvodynia, dysorgasmia, and testicular pain.

Dyspareunia refers to a condition where pain is experienced during sexual intercourse. This is predominantly a female complaint, but it does occur in males occasionally. Dyspareunia can have physical and/or emotional causes. The most common cause of pain during sex is inadequate vaginal lubrication (vaginal dryness) occurring from a lack of arousal, medications, or hormonal changes. Painful sex also can be a sign of illness, infection, cysts or tumors requiring medical treatment or surgery. Irritation from contraceptive creams and foams can also cause dryness, as can fear and anxiety about sex. An individual with dyspareunia experiences pain during sexual activity, has little or no interest in engaging in any type of sexual activity, and has a lack of sexual response.

Vaginismus refers to a condition where involuntary spasmodic muscle contractions occur at the entrance to the vagina, making anything entering the vagina painful. A sexual pain disorder that affects females, if sexual intercourse is attempted despite these contractions, a painful sexual experience results. Although the cause of vaginismus is currently unknown, both physical and psychological factors are suspected to play a role. An individual with vaginismus experiences pain during sexual activity, has little or no interest in engaging in any type of sexual activity, and has a lack of sexual response.

Vulvodynia (also known as vulvar vestibulitis) refers to a condition where pain is experienced in the vulva and includes pain outside the vulva on the labia or an itching, burning or sharp pain within the vulva. In this sexual pain disorder, a female will experience burning pain during sex which seems to be related to problems with the skin in the vulvar and vaginal areas. The cause of vulvodynia is currently unknown. Although the cause of vulvodynia is currently unknown, both physical and psychological factors are suspected to play a role. An individual with vulvodynia experiences pain during sexual activity, has little or no interest in engaging in any type of sexual activity, and has a lack of sexual response.

Dysorgasmia (also known as orgasmic pain) refers to a condition where pain is experienced during an orgasm. An individual with dysorgasmia experiences pain during sexual activity, has little or no interest in engaging in any type of sexual activity, and has a lack of sexual response.

Sexsomnia (also known as sleep sex) refers to a sexual dysfunction disorder where sexual behaviors are initiated and occur while an individual is asleep. Such behaviors can occur while alone, in the presence of a partner, or with a partner. Sexsomnia is in fact a type of sleep disorder (parasomnia) that involves sexual behaviors. Sexsomnia encompasses a range of sexual behaviors, including, but not limited to, sexual touching of another person, sexual vocalizations (moaning) and talking, masturbation (with and without orgasm), performing oral sex (with and without orgasm), and performing sexual intercourse (with and without orgasm). The current understanding is that the sexual behaviors are involuntary because the sleeping individual initiating them is unaware they are happening, and often has no memory of them occurring when confronted at a later point with the behavior.

Climacturia refers to a sexual dysfunction disorder where there is an involuntary release of urine at the time of orgasm during sexual activity. This disorder occurs in both males and females.

A composition or compound is administered to an individual. An individual comprises all mammals including a human being. Typically, any individual who is a candidate for a conventional sexual dysfunction disorder treatment is a candidate for a sexual dysfunction disorder treatment disclosed herein. Pre-operative evaluation typically includes routine history and physical examination in addition to thorough informed consent disclosing all relevant risks and benefits of the procedure.

With reference to a therapy comprising a TEM, the amount of a TEM disclosed herein used with the methods of treatment disclosed herein will typically be an effective amount. As used herein, the term “effective amount” is synonymous with “therapeutically effective amount”, “effective dose”, or “therapeutically effective dose” and when used in reference to treating a sexual dysfunction disorder means the minimum dose of a TEM alone necessary to achieve the desired therapeutic effect and includes a dose sufficient to reduce a symptom associated with a sexual dysfunction disorder. An effective amount refers to the total amount of a TEM administered to an individual in one setting. As such, an effective amount of a TEM does not refer to the amount administered per site. The effectiveness of a TEM disclosed herein in treating a sexual dysfunction disorder can be determined by observing an improvement in an individual based upon one or more clinical symptoms, and/or physiological indicators associated with the condition. An improvement in a sexual dysfunction disorder also can be indicated by a reduced need for a concurrent therapy.

With reference to a standard dose combination therapy comprising a Clostridial toxin and a TEM, an effective amount of a Clostridial toxin is one where in combination with a TEM the amount of a Clostridial toxin achieves the desired therapeutic effect. For example, typically about 75-150 U of BOTOX® (Allergan, Inc., Irvine, Calif.), a BoNT/A, is administered in order to treat a sexual dysfunction disorder.

With reference to a low dose combination therapy comprising a Clostridial toxin and a TEM, an effective amount of a Clostridial toxin is one where in combination with a TEM the amount of a Clostridial toxin achieves the desired therapeutic effect, but such an amount administered on its own would be ineffective. For example, typically about 75-150 U of BOTOX® (Allergan, Inc., Irvine, Calif.), a BoNT/A, is administered in order to treat a sexual dysfunction disorder. However, in a low dose combination therapy, a suboptimal effective amount of BoNT/A would be administered to treat a sexual dysfunction disorder when such toxin is used in a combined therapy with a TEM. For example, less that 50 U, less than 25 U, less than 15 U, less than 10 U, less than 7.5 U, less than 5 U, less than 2.5 U, or less than 1 U of BoNT/A would be administered to treat a sexual dysfunction disorder when used in a low dose combination therapy with a TEM as disclosed herein.

The appropriate effective amount of a Clostridial toxin and/or a TEM to be administered to an individual for a particular sexual dysfunction disorder can be determined by a person of ordinary skill in the art by taking into account factors, including, without limitation, the type of sexual dysfunction disorder, the location of the sexual dysfunction disorder, the cause of the sexual dysfunction disorder, the severity of the sexual dysfunction disorder, the degree of relief desired, the duration of relief desired, the particular TEM and/or Clostridial toxin used, the rate of excretion of the particular TEM and/or Clostridial toxin used, the pharmacodynamics of the particular TEM and/or Clostridial toxin used, the nature of the other compounds to be included in the composition, the particular route of administration, the particular characteristics, history and risk factors of the individual, such as, e.g., age, weight, general health and the like, or any combination thereof. Additionally, where repeated administration of a composition disclosed herein is used, an effective amount of a Clostridial toxin and/or a TEM will further depend upon factors, including, without limitation, the frequency of administration, the half-life of the particular TEM and/or Clostridial toxin used, or any combination thereof. In is known by a person of ordinary skill in the art that an effective amount of a composition comprising a Clostridial toxin and/or TEM can be extrapolated from in vitro assays and in vivo administration studies using animal models prior to administration to humans.

Wide variations in the necessary effective amount are to be expected in view of the differing efficiencies of the various routes of administration. For instance, oral administration generally would be expected to require higher dosage levels than administration by intravenous or intravitreal injection. Similarly, systemic administration of a TEM would be expected to require higher dosage levels than a local administration. Variations in these dosage levels can be adjusted using standard empirical routines of optimization, which are well-known to a person of ordinary skill in the art. The precise therapeutically effective dosage levels and patterns are preferably determined by the attending physician in consideration of the above-identified factors. One skilled in the art will recognize that the condition of the individual can be monitored throughout the course of therapy and that the effective amount of a TEM disclosed herein that is administered can be adjusted accordingly.

In aspects of this embodiment, a therapeutically effective amount of a composition comprising a TEM reduces a symptom associated with a sexual dysfunction disorder by, e.g., at least 10%, at least 20%, at least 30%, at least 40%, at least 50%, at least 60%, at least 70%, at least 80%, at least 90% or at least 100%. In other aspects of this embodiment, a therapeutically effective amount of a composition comprising a TEM reduces a symptom associated with a sexual dysfunction disorder by, e.g., at most 10%, at most 20%, at most 30%, at most 40%, at most 50%, at most 60%, at most 70%, at most 80%, at most 90% or at most 100%. In yet other aspects of this embodiment, a therapeutically effective amount of a composition comprising a TEM reduces a symptom associated with a sexual dysfunction disorder by, e.g., about 10% to about 100%, about 10% to about 90%, about 10% to about 80%, about 10% to about 70%, about 10% to about 60%, about 10% to about 50%, about 10% to about 40%, about 20% to about 100%, about 20% to about 90%, about 20% to about 80%, about 20% to about 20%, about 20% to about 60%, about 20% to about 50%, about 20% to about 40%, about 30% to about 100%, about 30% to about 90%, about 30% to about 80%, about 30% to about 70%, about 30% to about 60%, or about 30% to about 50%. In still other aspects of this embodiment, a therapeutically effective amount of the TEM is the dosage sufficient to inhibit neuronal activity for, e.g., at least one week, at least one month, at least two months, at least three months, at least four months, at least five months, at least six months, at least seven months, at least eight months, at least nine months, at least ten months, at least eleven months, or at least twelve months.

In other aspects of this embodiment, a therapeutically effective amount of a TEM generally is in the range of about 1 fg to about 3.0 mg. In aspects of this embodiment, an effective amount of a TEM can be, e.g., about 100 fg to about 3.0 mg, about 100 pg to about 3.0 mg, about 100 ng to about 3.0 mg, or about 100 μg to about 3.0 mg. In other aspects of this embodiment, an effective amount of a TEM can be, e.g., about 100 fg to about 750 μg, about 100 pg to about 750 μg, about 100 ng to about 750 μg, or about 1 μg to about 750 μg. In yet other aspects of this embodiment, a therapeutically effective amount of a TEM can be, e.g., at least 1 fg, at least 250 fg, at least 500 fg, at least 750 fg, at least 1 pg, at least 250 pg, at least 500 pg, at least 750 pg, at least 1 ng, at least 250 ng, at least 500 ng, at least 750 ng, at least 1 μg, at least 250 μg, at least 500 μg, at least 750 μg, or at least 1 mg. In still other aspects of this embodiment, a therapeutically effective amount of a composition comprising a TEM can be, e.g., at most 1 fg, at most 250 fg, at most 500 fg, at most 750 fg, at most 1 pg, at most 250 pg, at most 500 pg, at most 750 pg, at most 1 ng, at most 250 ng, at most 500 ng, at most 750 ng, at most 1 μg, at least 250 μg, at most 500 μg, at most 750 μg, or at most 1 mg.

In yet other aspects of this embodiment, a therapeutically effective amount of a TEM generally is in the range of about 0.00001 mg/kg to about 3.0 mg/kg. In aspects of this embodiment, an effective amount of a TEM can be, e.g., about 0.0001 mg/kg to about 0.001 mg/kg, about 0.03 mg/kg to about 3.0 mg/kg, about 0.1 mg/kg to about 3.0 mg/kg, or about 0.3 mg/kg to about 3.0 mg/kg. In yet other aspects of this embodiment, a therapeutically effective amount of a TEM can be, e.g., at least 0.00001 mg/kg, at least 0.0001 mg/kg, at least 0.001 mg/kg, at least 0.01 mg/kg, at least 0.1 mg/kg, or at least 1 mg/kg. In yet other aspects of this embodiment, a therapeutically effective amount of a TEM can be, e.g., at most 0.00001 mg/kg, at most 0.0001 mg/kg, at most 0.001 mg/kg, at most 0.01 mg/kg, at most 0.1 mg/kg, or at most 1 mg/kg.

In aspects of this embodiment, a therapeutically effective amount of a composition comprising a Clostridial toxin reduces a symptom associated with a sexual dysfunction disorder by, e.g., at least 10%, at least 20%, at least 30%, at least 40%, at least 50%, at least 60%, at least 70%, at least 80%, at least 90% or at least 100%. In other aspects of this embodiment, a therapeutically effective amount of a composition comprising a Clostridial toxin reduces a symptom associated with a sexual dysfunction disorder by, e.g., at most 10%, at most 20%, at most 30%, at most 40%, at most 50%, at most 60%, at most 70%, at most 80%, at most 90% or at most 100%. In yet other aspects of this embodiment, a therapeutically effective amount of a composition comprising a Clostridial toxin reduces a symptom associated with a sexual dysfunction disorder by, e.g., about 10% to about 100%, about 10% to about 90%, about 10% to about 80%, about 10% to about 70%, about 10% to about 60%, about 10% to about 50%, about 10% to about 40%, about 20% to about 100%, about 20% to about 90%, about 20% to about 80%, about 20% to about 20%, about 20% to about 60%, about 20% to about 50%, about 20% to about 40%, about 30% to about 100%, about 30% to about 90%, about 30% to about 80%, about 30% to about 70%, about 30% to about 60%, or about 30% to about 50%. In still other aspects of this embodiment, a therapeutically effective amount of a Clostridial toxin is the dosage sufficient to inhibit neuronal activity for, e.g., at least one week, at least one month, at least two months, at least three months, at least four months, at least five months, at least six months, at least seven months, at least eight months, at least nine months, at least ten months, at least eleven months, or at least twelve months.

In other aspects of this embodiment, a therapeutically effective amount of a Clostridial toxin generally is in the range of about 1 fg to about 30.0 μg. In other aspects of this embodiment, a therapeutically effective amount of a Clostridial toxin can be, e.g., at least 1.0 pg, at least 10 pg, at least 100 pg, at least 1.0 ng, at least 10 ng, at least 100 ng, at least 1.0 μg, at least 10 μg, at least 100 μg, or at least 1.0 mg. In still other aspects of this embodiment, a therapeutically effective amount of a Clostridial toxin can be, e.g., at most 1.0 pg, at most 10 pg, at most 100 pg, at most 1.0 ng, at most 10 ng, at most 100 ng, at most 1.0 μg, at most 10 μg, at most 100 μg, or at most 1.0 mg. In still other aspects of this embodiment, a therapeutically effective amount of a Clostridial toxin can be, e.g., about 1.0 pg to about 10 μg, about 10 pg to about 10 μg, about 100 pg to about 10 μg, about 1.0 ng to about 10 μg, about 10 ng to about 10 μg, or about 100 ng to about 10 μg. In still other aspects of this embodiment, a therapeutically effective amount of a Clostridial toxin can be from, e.g., about 1.0 pg to about 1.0 μg, about 10 pg to about 1.0 μg, about 100 pg to about 1.0 μg, about 1.0 ng to about 1.0 μg, about 10 ng to about 1.0 μg, or about 100 ng to about 1.0 μg. In other aspects of this embodiment, a therapeutically effective amount of a Clostridial toxin can be from, e.g., about 1.0 pg to about 100 ng, about 10 pg to about 100 ng, about 100 pg to about 100 ng, about 1.0 ng to about 100 ng, or about 10 ng to about 100 ng.

In yet other aspects of this embodiment, a therapeutically effective amount of a Clostridial toxin generally is in the range of about 0.1 U to about 2500 U. In other aspects of this embodiment, a therapeutically effective amount of a Clostridial toxin can be, e.g., at least 1.0 U, at least 10 U, at least 100 U, at least 250 U, at least 500 U, at least 750 U, at least 1,000 U, at least 1,500 U, at least 2,000 U, or at least 2,500 U. In still other aspects of this embodiment, a therapeutically effective amount of a Clostridial toxin can be, e.g., at most 1.0 U, at most 10 U, at most 100 U, at most 250 U, at most 500 U, at most 750 U, at most 1,000 U, at most 1,500 U, at most 2,000 U, or at most 2,500 U. In still other aspects of this embodiment, a therapeutically effective amount of a Clostridial toxin can be, e.g., about 1 U to about 2,000 U, about 10 U to about 2,000 U, about 50 U to about 2,000 U, about 100 U to about 2,000 U, about 500 U to about 2,000 U, about 1,000 U to about 2,000 U, about 1 U to about 1,000 U, about 10 U to about 1,000 U, about 50 U to about 1,000 U, about 100 U to about 1,000 U, about 500 U to about 1,000 U, about 1 U to about 500 U, about 10 U to about 500 U, about 50 U to about 500 U, about 100 U to about 500 U, about 1 U to about 100 U, about 10 U to about 100 U, about 50 U to about 100 U, about 0.1 U to about 1 U, about 0.1 U to about 5 U, about 0.1 U to about 10 U, about 0.1 U to about 15 U, about 0.1 U to about 20 U, about 0.1 U to about 25 U.

In still other aspects of this embodiment, a therapeutically effective amount of a Clostridial toxin generally is in the range of about 0.0001 U/kg to about 3,000 U/kg. In aspects of this embodiment, a therapeutically effective amount of a Clostridial toxin can be, e.g., at least 0.001 U/kg, at least 0.01 U/kg, at least 0.1 U/kg, at least 1.0 U/kg, at least 10 U/kg, at least 100 U/kg, or at least 1000 U/kg. In other aspects of this embodiment, a therapeutically effective amount of a Clostridial toxin can be, e.g., at most 0.001 U/kg, at most 0.01 U/kg, at most 0.1 U/kg, at most 1.0 U/kg, at most 10 U/kg, at most 100 U/kg, or at most 1000 U/kg. In yet other aspects of this embodiment, a therapeutically effective amount of a Clostridial toxin can be between, e.g., about 0.001 U/kg to about 1 U/kg, about 0.01 U/kg to about 1 U/kg, about 0.1 U/kg to about 1 U/kg, about 0.001 U/kg to about 10 U/kg, about 0.01 U/kg to about 10 U/kg, about 0.1 U/kg to about 10 U/kg about 1 U/kg to about 10 U/kg, about 0.001 U/kg to about 100 U/kg, about 0.01 U/kg to about 100 U/kg, about 0.1 U/kg to about 100 U/kg, about 1 U/kg to about 100 U/kg, or about 10 U/kg to about 100 U/kg. As used herein, the term “unit” or “U” is refers to the LD₅₀ dose, which is defined as the amount of a Clostridial toxin disclosed herein that killed 50% of the mice injected with the Clostridial toxin.

In aspects of this embodiment, a therapeutically effective amount of a standard or low combination therapy comprising a Clostridial toxin and a TEM reduces a symptom associated with a sexual dysfunction disorder by, e.g., at least 10%, at least 20%, at least 30%, at least 40%, at least 50%, at least 60%, at least 70%, at least 80%, at least 90% or at least 100%. In other aspects of this embodiment, a therapeutically effective amount of a standard or low combination therapy comprising a Clostridial toxin and a TEM reduces a symptom associated with a sexual dysfunction disorder by, e.g., at most 10%, at most 20%, at most 30%, at most 40%, at most 50%, at most 60%, at most 70%, at most 80%, at most 90% or at most 100%. In yet other aspects of this embodiment, a therapeutically effective amount of a standard or low combination therapy comprising a Clostridial toxin and a TEM reduces a symptom associated with a sexual dysfunction disorder by, e.g., about 10% to about 100%, about 10% to about 90%, about 10% to about 80%, about 10% to about 70%, about 10% to about 60%, about 10% to about 50%, about 10% to about 40%, about 20% to about 100%, about 20% to about 90%, about 20% to about 80%, about 20% to about 20%, about 20% to about 60%, about 20% to about 50%, about 20% to about 40%, about 30% to about 100%, about 30% to about 90%, about 30% to about 80%, about 30% to about 70%, about 30% to about 60%, or about 30% to about 50%. In still other aspects of this embodiment, a therapeutically effective amount of a standard or low combination therapy comprising a Clostridial toxin and a TEM is the dosage sufficient to inhibit neuronal activity for, e.g., at least one week, at least one month, at least two months, at least three months, at least four months, at least five months, at least six months, at least seven months, at least eight months, at least nine months, at least ten months, at least eleven months, or at least twelve months.

In other aspects of this embodiment, a therapeutically effective amount of a standard or low combination therapy comprising a Clostridial toxin and a TEM generally is in a Clostridial toxin: TEM molar ratio of about 1:1 to about 1:10,000. In other aspects of this embodiment, a therapeutically effective amount of a standard or low combination therapy comprising a Clostridial toxin and a TEM can be in a Clostridial toxin: TEM molar ratio of, e.g., about 1:1, about 1:2, about 1:5, about 1:10, about 1:25, about 1:50, about 1:75, about 1:100, about 1:200, about 1:300, about 1:400, about 1:500, about 1:600, about 1:700, about 1:800, about 1:900, about 1:1000, about 1:2000, about 1:3000, about 1:4000, about 1:5000, about 1:6000, about 1:7000, about 1:8000, about 1:9000, or about 1:10,000. In yet other aspects of this embodiment, a therapeutically effective amount of standard or low combination therapy comprising a Clostridial toxin and a TEM can be in a Clostridial toxin: TEM molar ratio of, e.g., at least 1:1, at least 1:2, at least 1:5, at least 1:10, at least 1:25, at least 1:50, at least 1:75, at least 1:100, at least 1:200, at least 1:300, at least 1:400, at least 1:500, at least 1:600, at least 1:700, at least 1:800, at least 1:900, at least 1:1000, at least 1:2000, at least 1:3000, at least 1:4000, at least 1:5000, at least 1:6000, at least 1:7000, at least 1:8000, at least 1:9000, or at least 1:10,000. In still other aspects of this embodiment, a therapeutically effective amount of a standard or low combination therapy comprising a Clostridial toxin and a TEM can be in a Clostridial toxin: TEM molar ratio of between, e.g., about 1:1 to about 1:10,000, about 1:10 to about 1:10,000, about 1:100 to about 1:10,000, about 1:500 to about 1:10,000, about 1:1000 to about 1:10,000, about 1:5000 to about 1:10,000, about 1:1 to about 1:1000, about 1:10 to about 1:1000, about 1:100 to about 1:1000, about 1:250 to about 1:1000, about 1:500 to about 1:1000, about 1:750 to about 1:1000, about 1:1 to about 1:500, about 1:10 to about 1:500, about 1:50 to about 1:500, about 1:100 to about 1:500, about 1:250 to about 1:500, about 1:1 to about 1:100, about 1:10 to about 1:100, about 1:25 to about 1:100, about 1:50 to about 1:100, or about 1:75 to about 1:100.

In yet other aspects of this embodiment, a therapeutically effective amount of a standard combination therapy comprising a Clostridial toxin and a TEM generally is in a range of about 0.50 U to about 250 U of Clostridial toxin and about 0.1 μg to about 2,000.0 μg of a TEM. In aspects of this embodiment, a therapeutically effective amount of a combined therapy comprising a Clostridial toxin and a TEM can be, e.g., about 0.1 U to about 10 U of a Clostridial toxin and about 10 μg to about 1,000 μg of a TEM, about 0.1 U to about 10 U of a Clostridial toxin and about 10 μg to about 500 μg of a TEM, about 0.1 U to about 10 U of a Clostridial toxin and about 10 μg to about 100 μg of a TEM, about 0.5 U to about 10 U of a Clostridial toxin and about 10 μg to about 1,000 μg of a TEM, about 0.5 U to about 10 U of a Clostridial toxin and about 10 μg to about 500 μg of a TEM, about 0.5 U to about 10 U of a Clostridial toxin and about 10 μg to about 100 μg of a TEM, about 1 U to about 10 U of a Clostridial toxin and about 100 μg to about 1,000 μg of a TEM, about 1 U to about 10 U of a Clostridial toxin and about 100 μg to about 500 μg of a TEM, or about 1 U to about 10 U of a Clostridial toxin and about 100 μg to about 100 μg of a TEM.

In yet other aspects of this embodiment, a therapeutically effective amount of a low combination therapy comprising a Clostridial toxin and a TEM generally is in a range of about 0.01 U to about 50 U of Clostridial toxin and about 0.1 μg to about 2,000.0 μg of a TEM. In aspects of this embodiment, a therapeutically effective amount of a combined therapy comprising a Clostridial toxin and a TEM can be, e.g., about 0.1 U to about 10 U of a Clostridial toxin and about 10 μg to about 1,000 μg of a TEM, about 0.1 U to about 10 U of a Clostridial toxin and about 10 μg to about 500 μg of a TEM, about 0.1 U to about 10 U of a Clostridial toxin and about 10 μg to about 100 μg of a TEM, about 0.5 U to about 10 U of a Clostridial toxin and about 10 μg to about 1,000 μg of a TEM, about 0.5 U to about 10 U of a Clostridial toxin and about 10 μg to about 500 μg of a TEM, about 0.5 U to about 10 U of a Clostridial toxin and about 10 μg to about 100 μg of a TEM, about 1 U to about 10 U of a Clostridial toxin and about 100 μg to about 1,000 μg of a TEM, about 1 U to about 10 U of a Clostridial toxin and about 100 μg to about 500 μg of a TEM, or about 1 U to about 10 U of a Clostridial toxin and about 100 μg to about 100 μg of a TEM.

Dosing can be single dosage or cumulative (serial dosing), and can be readily determined by one skilled in the art. For instance, treatment of a sexual dysfunction disorder may comprise a one-time administration of an effective dose of a composition disclosed herein. As a non-limiting example, an effective dose of a composition disclosed herein can be administered once to an individual, e.g., as a single injection or deposition at or near the site exhibiting a symptom of a sexual dysfunction disorder. Alternatively, treatment of a sexual dysfunction disorder may comprise multiple administrations of an effective dose of a composition disclosed herein carried out over a range of time periods, such as, e.g., daily, once every few days, weekly, monthly or yearly. As a non-limiting example, a composition disclosed herein can be administered once or twice yearly to an individual. The timing of administration can vary from individual to individual, depending upon such factors as the severity of an individual's symptoms. For example, an effective dose of a composition disclosed herein can be administered to an individual once a month for an indefinite period of time, or until the individual no longer requires therapy. A person of ordinary skill in the art will recognize that the condition of the individual can be monitored throughout the course of treatment and that the effective amount of a composition disclosed herein that is administered can be adjusted accordingly.

A composition disclosed herein can be administered to an individual using a variety of routes. Routes of administration suitable for a method of treating a sexual dysfunction disorder as disclosed herein include both local and systemic administration. Local administration results in significantly more delivery of a composition to a specific location as compared to the entire body of the individual, whereas, systemic administration results in delivery of a composition to essentially the entire body of the individual. Routes of administration suitable for a method of treating a sexual dysfunction disorder as disclosed herein also include both central and peripheral administration. Central administration results in delivery of a composition to essentially the central nervous system of an individual and includes, e.g., intrathecal administration, epidural administration as well as a cranial injection or implant. Peripheral administration results in delivery of a composition to essentially any area of an individual outside of the central nervous system and encompasses any route of administration other than direct administration to the spine or brain. The actual route of administration of a composition disclosed herein used can be determined by a person of ordinary skill in the art by taking into account factors, including, without limitation, the type of sexual dysfunction disorder, the location of the sexual dysfunction disorder, the cause of the sexual dysfunction disorder, the severity of the sexual dysfunction disorder, the degree of relief desired, the duration of relief desired, the particular Clostridial toxin and/or TEM used, the rate of excretion of the Clostridial toxin and/or TEM used, the pharmacodynamics of the Clostridial toxin and/or TEM used, the nature of the other compounds to be included in the composition, the particular route of administration, the particular characteristics, history and risk factors of the individual, such as, e.g., age, weight, general health and the like, or any combination thereof.

In an embodiment, a composition disclosed herein is administered systemically to an individual. In another embodiment, a composition disclosed herein is administered locally to an individual. In an aspect of this embodiment, a composition disclosed herein is administered to a nerve of an individual. In another aspect of this embodiment, a composition disclosed herein is administered to the area surrounding a nerve of an individual.

A composition disclosed herein can be administered to an individual using a variety of delivery mechanisms. The actual delivery mechanism used to administer a composition disclosed herein to an individual can be determined by a person of ordinary skill in the art by taking into account factors, including, without limitation, the type of sexual dysfunction disorder, the location of the sexual dysfunction disorder, the cause of the sexual dysfunction disorder, the severity of the sexual dysfunction disorder, the degree of relief desired, the duration of relief desired, the particular Clostridial toxin and/or TEM used, the rate of excretion of the Clostridial toxin and/or TEM used, the pharmacodynamics of the Clostridial toxin and/or TEM used, the nature of the other compounds to be included in the composition, the particular route of administration, the particular characteristics, history and risk factors of the individual, such as, e.g., age, weight, general health and the like, or any combination thereof.

In an embodiment, a composition disclosed herein is administered by injection. In aspects of this embodiment, administration of a composition disclosed herein is by, e.g., intramuscular injection, intraorgan injection, subdermal injection, dermal injection, intracranical injection, spinal injection, or injection into any other body area for the effective administration of a composition disclosed herein. In aspects of this embodiment, injection of a composition disclosed herein is to a nerve or into the area surrounding a nerve.

In another embodiment, a composition disclosed herein is administered by catheter. In aspects of this embodiment, administration of a composition disclosed herein is by, e.g., a catheter placed in an epidural space.

A composition disclosed herein as disclosed herein can also be administered to an individual in combination with other therapeutic compounds to increase the overall therapeutic effect of the treatment. The use of multiple compounds to treat an indication can increase the beneficial effects while reducing the presence of side effects.

Aspects of the present invention can also be described as follows:
1. A method of treating a sexual dysfunction disorder in an individual, the method comprising the step of administering to the individual in need thereof a therapeutically effective amount of a composition including a TEM, wherein administration of the composition reduces a symptom of the sexual dysfunction disorder, thereby treating the individual.
2. A use of a TEM in the manufacturing a medicament for treating a sexual dysfunction disorder in an individual in need thereof.
3. A use of a TEM in the treatment of a sexual dysfunction disorder in an individual in need thereof.
4. A method of treating a sexual dysfunction disorder in an individual, the method comprising the step of administering to the individual in need thereof a therapeutically effective amount of a composition including a Clostridial neurotoxin and a TEM, wherein administration of the composition reduces a symptom of the sexual dysfunction disorder, thereby treating the individual.
5. A use of a Clostridial neurotoxin and a TEM in the manufacturing a medicament for treating a sexual dysfunction disorder in an individual in need thereof.
6. A use of a Clostridial neurotoxin and a TEM in the treatment of a sexual dysfunction disorder in an individual in need thereof.
7. The embodiments of 1 to 6, wherein the TEM comprises a linear amino-to-carboxyl single polypeptide order of 1) a Clostridial toxin enzymatic domain, a Clostridial toxin translocation domain, a targeting domain, 2) a Clostridial toxin enzymatic domain, a targeting domain, a Clostridial toxin translocation domain, 3) a targeting domain, a Clostridial toxin translocation domain, and a Clostridial toxin enzymatic domain, 4) a targeting domain, a Clostridial toxin enzymatic domain, a Clostridial toxin translocation domain, 5) a Clostridial toxin translocation domain, a Clostridial toxin enzymatic domain and a targeting domain, or 6) a Clostridial toxin translocation domain, a targeting domain and a Clostridial toxin enzymatic domain.
8. The embodiments of 1 to 6, wherein the TEM comprises a linear amino-to-carboxyl single polypeptide order of 1) a Clostridial toxin enzymatic domain, an exogenous protease cleavage site, a Clostridial toxin translocation domain, a targeting domain, 2) a Clostridial toxin enzymatic domain, an exogenous protease cleavage site, a targeting domain, a Clostridial toxin translocation domain, 3) a targeting domain, a Clostridial toxin translocation domain, an exogenous protease cleavage site and a Clostridial toxin enzymatic domain, 4) a targeting domain, a Clostridial toxin enzymatic domain, an exogenous protease cleavage site, a Clostridial toxin translocation domain, 5) a Clostridial toxin translocation domain, an exogenous protease cleavage site, a Clostridial toxin enzymatic domain and a targeting domain, or 6) a Clostridial toxin translocation domain, an exogenous protease cleavage site, a targeting domain and a Clostridial toxin enzymatic domain.
9. The embodiments of 1 to 8, wherein the Clostridial toxin translocation domain is a BoNT/A translocation domain, a BoNT/B translocation domain, a BoNT/C1 translocation domain, a BoNT/D translocation domain, a BoNT/E translocation domain, a BoNT/F translocation domain, a BoNT/G translocation domain, a TeNT translocation domain, a BaNT translocation domain, or a BuNT translocation domain.
10. The embodiments of 1 to 9, wherein the Clostridial toxin enzymatic domain is a BoNT/A enzymatic domain, a BoNT/B enzymatic domain, a BoNT/C1 enzymatic domain, a BoNT/D enzymatic domain, a BoNT/E enzymatic domain, a BoNT/F enzymatic domain, a BoNT/G enzymatic domain, a TeNT enzymatic domain, a BaNT enzymatic domain, or a BuNT enzymatic domain.
11. The embodiments of 1 to 10, wherein the targeting domain is a sensory neuron targeting domain, a sympathetic neuron targeting domain, or a parasympathetic neuron targeting domain.
12. The embodiments of 1 to 10, wherein the targeting domain is an opioid peptide targeting domain, a galanin peptide targeting domain, a PAR peptide targeting domain, a somatostatin peptide targeting domain, a neurotensin peptide targeting domain, a SLURP peptide targeting domain, an angiotensin peptide targeting domain, a tachykinin peptide targeting domain, a Neuropeptide Y related peptide targeting domain, a kinin peptide targeting domain, a melanocortin peptide targeting domain, or a granin peptide targeting domain, a glucagon like hormone peptide targeting domain, a secretin peptide targeting domain, a pituitary adenylate cyclase activating peptide (PACAP) peptide targeting domain, a growth hormone-releasing hormone (GHRH) peptide targeting domain, a vasoactive intestinal peptide (VIP) peptide targeting domain, a gastric inhibitory peptide (GIP) peptide targeting domain, a calcitonin peptide targeting domain, a visceral gut peptide targeting domain, a neurotrophin peptide targeting domain, a head activator (HA) peptide, a glial cell line-derived neurotrophic factor (GDNF) family of ligands (GFL) peptide targeting domain, a RF-amide related peptide (RFRP) peptide targeting domain, a neurohormone peptide targeting domain, or a neuroregulatory cytokine peptide targeting domain, an interleukin (IL) targeting domain, vascular endothelial growth factor (VEGF) targeting domain, an insulin-like growth factor (IGF) targeting domain, an epidermal growth factor (EGF) targeting domain, a Transformation Growth Factor-β (TGFβ) targeting domain, a Bone Morphogenetic Protein (BMP) targeting domain, a Growth and Differentiation Factor (GDF) targeting domain, an activin targeting domain, or a Fibroblast Growth Factor (FGF) targeting domain, or a Platelet-Derived Growth Factor (PDGF) targeting domain.
13. The embodiments of 8 to 12, wherein the exogenous protease cleavage site is a plant papain cleavage site, an insect papain cleavage site, a crustacian papain cleavage site, an enterokinase cleavage site, a human rhinovirus 3C protease cleavage site, a human enterovirus 3C protease cleavage site, a tobacco etch virus protease cleavage site, a Tobacco Vein Mottling Virus cleavage site, a subtilisin cleavage site, a hydroxylamine cleavage site, or a Caspase 3 cleavage site.
14. The embodiments of 1 to 13, wherein the Clostridial neurotoxin is a BoNT/A, a BoNT/B, a BoNT/C1, a BoNT/D, a BoNT/E, a BoNT/F, a BoNT/G, a TeNT, a BaNT, a BuNT, or any combination thereof.
15. The embodiments of 1 to 14, wherein the sexual dysfunction disorder is a sexual desire disorder, a sexual arousal disorder, a sexual orgasm disorder, a sexual pain disorder, a sexsomnia, or a climacturia.
16. The embodiment of 15, wherein the sexual desire disorder is a hypoactive sexual desire disorder, a sexual aversion disorder, or a hyperactive sexual desire disorder.
17. The embodiment of 15, wherein the sexual arousal disorder is a deficient sexual arousal disorder, a persistent sexual arousal disorder, or a priapism.
18. The embodiment of 15, wherein the sexual orgasm disorder is a male orgasmic disorder, a male anorgasmia, a premature ejaculation, an ejaculatory incompetence, a female orgasmic disorder, a female anorgasmia, or an inhibited female orgasm.
19. The embodiment of 15, wherein the sexual pain disorder is a dyspareunia, a vaginismus, a vulvodynia, a dysorgasmia, or a testicular pain.

### EXAMPLES

The following non-limiting examples are provided for illustrative purposes only in order to facilitate a more complete understanding of representative embodiments now contemplated. These examples should not be construed to limit any of the embodiments described in the present specification, including those pertaining to the compounds, compositions, methods or uses of treating a sexual dysfunction disorder.

### Example 1

### Treatment of a Sexual Desire Disorder

A female complains of a lack of sexual thoughts and a chronically reduced desire to engage in sexual activity with her husband. After routine history and physical examination, a physician diagnosis the patient with a hypoactive sexual desire disorder involving abnormal sensory neuron activity and identifies the nerves and/or muscles involved in the condition. The woman is treated by injecting a composition comprising a TEM as disclosed in the present specification. The patient's condition is monitored and after about 2 weeks from treatment, the woman indicates that she has had sexual feelings and thoughts for her husband and that they have engaged in sexual activity. At one and three month check-ups, the woman indicates that she continues to have increased improvement and indicates that her relationship with her husband is very satisfying. This increase in sexual thoughts and activity indicates a successful treatment with the composition comprising a TEM.

A male complains about being unusually apprehensive or repulsed by the thought of engaging in any type of sexual thoughts, fantasies or activity with his wife of 30 years. After routine history and physical examination, a physician diagnosis the patient with a sexual aversion disorder involving abnormal sensory neuron activity and identifies the nerves and/or muscles involved in the condition. The man is treated by injecting a composition comprising a TEM as disclosed in the present specification. The patient's condition is monitored and after about 2 weeks from treatment, the man indicates that he is once again attracted to his wife and they have engaged in sexual activity. At two and four month check-ups, the man indicates that he continues to engage in sexual thoughts about his wife and that they routinely engage in sexual activity. This increase in sexual thoughts and activity indicates a successful treatment with the composition comprising a TEM.

A man complains about always having sexual thoughts and acting out on those thought and this behavior it interfering with his work and social life. After routine history and physical examination, a physician diagnosis the patient with a hyperactive sexual desire disorder involving abnormal sensory neuron activity and identifies the nerves and/or muscles involved in the condition. The man is treated by injecting a composition comprising a TEM as disclosed in the present specification. The patient's condition is monitored and after about 2 weeks from treatment, the man indicates he is not having as many sexual fantasies as before and can get through a day without acting out. At two and four month check-ups, the man indicates that he continues to not be preoccupied with sex and sexual fantasies and is more satisfied with his life. This decrease in sexual fantasies and acting out indicates a successful treatment with the composition comprising a TEM.

### Example 2

### Treatment of a Sexual Arousal Disorder

A female complains that she is experiencing a lack of an arousal response even after sexual stimulation by her husband. After routine history and physical examination, a physician diagnosis the patient with a deficient sexual arousal disorder involving abnormal sensory neuron activity and identifies the nerves and/or muscles involved in the condition. The woman is treated by injecting a composition comprising a TEM as disclosed in the present specification. The patient's condition is monitored and after about 2 weeks from treatment, the woman indicates that she now becomes sexually aroused when she engages in sexual activity with her husband. At one, three and five month check-ups, the woman indicates that she continues to become aroused during sexual activity and indicates that her relationship with her husband is very satisfying. This increase in sexual arousal and activity indicates a successful treatment with the composition comprising a TEM.

A man complains about always having spontaneous, persistent, and uncontrollable genital arousal in the absence of any sexual stimulation and this behavior it interfering with his work and social life. After routine history and physical examination, a physician diagnosis the patient with a persistent sexual arousal disorder involving abnormal sensory neuron activity and identifies the nerves and/or muscles involved in the condition. The man is treated by injecting a composition comprising a TEM as disclosed in the present specification. The patient's condition is monitored and after about 2 weeks from treatment, the man indicates he is not having as many of these abnormal sexual arousal incidents as before. At two and four month check-ups, the man indicates that he continues to not have any spontaneous, persistent, and uncontrollable genital arousal in the absence of any sexual stimulation. This decrease in abnormal sexual arousal indicates a successful treatment with the composition comprising a TEM.

A man complains that he is experiencing a persistent penile erection that last for up to four hours. After routine history and physical examination, a physician diagnosis the patient with a priapism involving abnormal sensory neuron activity. The man is treated by injecting a composition comprising a TEM as disclosed in the present specification into the sensory rami. The patient's condition is monitored and after about 2 weeks from treatment, the man indicates that he is experiences detumescence shortly after ejaculation. At one, three and five month check-ups, the man indicates that he continues to have normal detumescence. This return to normal detumescence indicates a successful treatment with the composition comprising a TEM.

### Example 3

### Treatment of a Sexual Orgasm Disorder

A man complains about having a delay or absence in ejaculation, even after sufficient sexual stimulation and arousal. After routine history and physical examination, a physician diagnosis the patient with an ejaculatory incompetence disorder involving abnormal sensory neuron activity and identifies the nerves and/or muscles involved in the condition. The man is treated by injecting a composition comprising a TEM as disclosed in the present specification. The patient's condition is monitored and after about 2 weeks from treatment, the man indicates he is able to better control his ejaculations. At two and four month check-ups, the man indicates that he continues to have better control of his ejaculations as compared to before the treatment. This increase in ejaculation control indicates a successful treatment with the composition comprising a TEM.

A man complains about unable to control ejaculation so that it occurs before satisfying sexual relations can take place with the wife. After routine history and physical examination, a physician diagnosis the patient with a premature ejaculation disorder involving abnormal sensory neuron activity and identifies the nerves and/or muscles involved in the condition. The patient's condition is monitored and after about 2 weeks from treatment, the man indicates he is experiencing ejaculations at the appropriate time and manner. At two and four month check-ups, the man indicates that he continues to experience ejaculations after sufficient sexual stimulation and arousal. This increase in appropriate ejaculation response indicates a successful treatment with the composition comprising a TEM. A man suffering from a male anorgasmia can be treated in a similar manner.

A woman complains about not experiencing an orgasm even after sufficient sexual stimulation and arousal. After routine history and physical examination, a physician diagnosis the patient with a female anorgasmia disorder involving abnormal sensory neuron activity and identifies the nerves and/or muscles involved in the condition. The woman is treated by injecting a composition comprising a TEM as disclosed in the present specification. The patient's condition is monitored and after about 2 weeks from treatment, the woman indicates she is now experiencing orgasms again when she engages in sexual activity with her husband. At two and four month check-ups, the woman indicates that she continues to have orgasms and is very satisfied with her sex life. This increase in orgasms after sufficient sexual stimulation and arousal indicates a successful treatment with the composition comprising a TEM.

A woman complains about having a delay or absence in reaching orgasm after sufficient sexual stimulation and arousal. After routine history and physical examination, a physician diagnosis the patient with an inhibited female orgasm disorder involving abnormal sensory neuron activity and identifies the nerves and/or muscles involved in the condition. The woman is treated by injecting a composition comprising a TEM as disclosed in the present specification. The patient's condition is monitored and after about 2 weeks from treatment, the woman indicates she can now reach orgasm during sexual activity with her husband. At two and four month check-ups, the woman indicates that she continues to have orgasms. This increase in reaching orgasms after sufficient sexual stimulation and arousal indicates a successful treatment with the composition comprising a TEM.

### Example 4

### Treatment of a Sexual Pain Disorder

A woman complains about having spasmodic vaginal muscle contractions that make engaging in sexual activity painful. After routine history and physical examination, a physician diagnosis the patient with a vaginismus disorder involving abnormal sensory neuron activity and identifies the nerves and/or muscles involved in the condition. The woman is treated by injecting a composition comprising a TEM as disclosed in the present specification. The patient's condition is monitored and after about 2 weeks from treatment, the woman indicates she is experiencing less vaginal muscle contractions and when such contractions occur, they are not as intense. This reduction in muscle contractions has enabled her to engage in sexual activity with her husband without experiencing pain. At two and four month check-ups, the woman indicates that the spasmodic vaginal muscle contractions have gone away. This reduction in spasmodic vaginal muscle contractions indicates a successful treatment with the composition comprising a TEM. A similar type of treatment regime can be employed for a person suffering from dyspareunia, vulvodynia, dysorgasmia, or testicular pain.

### Example 5

### Treatment of a Sexsominia

A man complains about initiating sexual behaviors while he is asleep. After routine history and physical examination, a physician diagnosis the patient with a sexsomnia disorder involving abnormal sensory neuron activity and identifies the nerves and/or muscles involved in the condition. The man is treated by injection of a composition comprising a TEM as disclosed in the present specification, targeting the sensory rami. The patient's condition is monitored and after about 2 weeks from treatment, the man indicates he has not initiated any sexual behaviors while sleeping. At two and four month check-ups, the man indicates that he continues to sleep through the night without initiating any sexual behaviors. This decrease in initiating sexual behaviors while asleep indicates a successful treatment with the composition comprising a TEM.

### Example 6

### Treatment of a Climacturia

A woman complains of involuntarily releasing urine at the time of orgasm during sexual activity. After routine history and physical examination, a physician diagnosis the patient with a climacturia disorder involving abnormal sensory neuron activity and identifies the nerves and/or muscles involved in the condition. The woman is treated by injecting a composition comprising a TEM as disclosed in the present specification. The patient's condition is monitored and after about 2 weeks from treatment, the woman indicates she can now reach orgasm without urinating. At two and four month check-ups, the woman indicates that she continues to have normal orgasms and sexual activity. This decrease in urination during sexual activity indicates a successful treatment with the composition comprising a TEM.

A man complains of involuntarily releasing urine at the time of ejaculation during sexual activity. After routine history and physical examination, a physician diagnosis the patient with a climacturia disorder involving abnormal sensory neuron activity and identifies the nerves and/or muscles involved in the condition. The man is treated by intradermal injection of a composition comprising a TEM as disclosed in the present specification, targeting the prostate. The patient's condition is monitored and after about 2 weeks from treatment, the man indicates he can now ejaculate without urinating. At two and four month check-ups, the man indicates that he continues to ejaculate without urinating. This decrease in urination during sexual activity indicates a successful treatment with the composition comprising a TEM.

### CONCLUSION

In closing, it is to be understood that although aspects of the present specification are highlighted by referring to specific embodiments, one skilled in the art will readily appreciate that these disclosed embodiments are only illustrative of the principles of the subject matter disclosed herein. Therefore, it should be understood that the disclosed subject matter is in no way limited to a particular methodology, protocol, and/or reagent, etc., described herein. As such, various modifications or changes to or alternative configurations of the disclosed subject matter can be made in accordance with the teachings herein without departing from the spirit of the present specification. Lastly, the terminology used herein is for the purpose of describing particular embodiments only, and is not intended to limit the scope of the present invention, which is defined solely by the claims. Accordingly, the present invention is not limited to that precisely as shown and described.

Certain embodiments of the present invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the present invention to be practiced otherwise than specifically described herein. Accordingly, this invention includes all modifications and equivalents of the subject matter recited in the claims appended hereto as permitted by applicable law. Moreover, any combination of the above-described embodiments in all possible variations thereof is encompassed by the invention unless otherwise indicated herein or otherwise clearly contradicted by context.

Groupings of alternative embodiments, elements, or steps of the present invention are not to be construed as limitations. Each group member may be referred to and claimed individually or in any combination with other group members disclosed herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Unless otherwise indicated, all numbers expressing a characteristic, item, quantity, parameter, property, term, and so forth used in the present specification and claims are to be understood as being modified in all instances by the term “about.” As used herein, the term “about” means that the characteristic, item, quantity, parameter, property, or term so qualified encompasses a range of plus or minus ten percent above and below the value of the stated characteristic, item, quantity, parameter, property, or term. Accordingly, unless indicated to the contrary, the numerical parameters set forth in the specification and attached claims are approximations that may vary. At the very least, and not as an attempt to limit the application of the doctrine of equivalents to the scope of the claims, each numerical indication should at least be construed in light of the number of reported significant digits and by applying ordinary rounding techniques. Notwithstanding that the numerical ranges and values setting forth the broad scope of the invention are approximations, the numerical ranges and values set forth in the specific examples are reported as precisely as possible. Any numerical range or value, however, inherently contains certain errors necessarily resulting from the standard deviation found in their respective testing measurements. Recitation of numerical ranges of values herein is merely intended to serve as a shorthand method of referring individually to each separate numerical value falling within the range. Unless otherwise indicated herein, each individual value of a numerical range is incorporated into the present specification as if it were individually recited herein.

The terms “a,” “an,” “the” and similar referents used in the context of describing the present invention (especially in the context of the following claims) are to be construed to cover both the singular and the plural, unless otherwise indicated herein or clearly contradicted by context. All methods described herein can be performed in any suitable order unless otherwise indicated herein or otherwise clearly contradicted by context. The use of any and all examples, or exemplary language (e.g., “such as”) provided herein is intended merely to better illuminate the present invention and does not pose a limitation on the scope of the invention otherwise claimed. No language in the present specification should be construed as indicating any non-claimed element essential to the practice of the invention.

Specific embodiments disclosed herein may be further limited in the claims using consisting of or consisting essentially of language. When used in the claims, whether as filed or added per amendment, the transition term “consisting of” excludes any element, step, or ingredient not specified in the claims. The transition term “consisting essentially of” limits the scope of a claim to the specified materials or steps and those that do not materially affect the basic and novel characteristic(s). Embodiments of the present invention so claimed are inherently or expressly described and enabled herein.

All patents, patent publications, and other publications referenced and identified in the present specification are individually and expressly incorporated herein by reference in their entirety for the purpose of describing and disclosing, for example, the compositions and methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

1. A method of treating a sexual dysfunction disorder in an individual, the method comprising the step of administering to the individual in need thereof a therapeutically effective amount of a composition including a TEM comprising a targeting domain, a Clostridial toxin translocation domain and a Clostridial toxin enzymatic domain, wherein the targeting domain is a sensory neuron targeting domain, a sympathetic neuron targeting domain, or a parasympathetic neuron targeting domain, and wherein administration of the composition reduces a symptom of the sexual dysfunction disorder, thereby treating the individual.

2. The method of claim 1, wherein the sexual dysfunction disorder is a sexual desire disorder, a sexual arousal disorder, a sexual orgasm disorder, a sexual pain disorder, a sexsomnia, or a climacturia.

3. The method of claim 2, wherein the sexual desire disorder is a hypoactive sexual desire disorder, a sexual aversion disorder, or a hyperactive sexual desire disorder.

4. The method of claim 2, wherein the sexual arousal disorder is a deficient sexual arousal disorder, a persistent sexual arousal disorder, or a priapism.

5. The method of claim 2, wherein the sexual orgasm disorder is a male orgasmic disorder, a male anorgasmia, a premature ejaculation, an ejaculatory incompetence, a female orgasmic disorder, a female anorgasmia, or an inhibited female orgasm.

6. The method of claim 2, wherein the sexual pain disorder is a dyspareunia, a vaginismus, a vulvodynia, a dysorgasmia, or a testicular pain.

7. A method of treating a sexual dysfunction disorder in an individual, the method comprising the step of administering to the individual in need thereof a therapeutically effective amount of a composition including a TEM comprising a targeting domain, a Clostridial toxin translocation domain, a Clostridial toxin enzymatic domain, and an exogenous protease cleavage site, wherein the targeting domain is a sensory neuron targeting domain, a sympathetic neuron targeting domain, or a parasympathetic neuron targeting domain, and wherein administration of the composition reduces a symptom of the sexual dysfunction disorder, thereby treating the individual.

8. The method of claim 7, wherein the TEM comprises a linear amino-to-carboxyl single polypeptide order of 1) the Clostridial toxin enzymatic domain, the exogenous protease cleavage site, the Clostridial toxin translocation domain, the targeting domain, 2) the Clostridial toxin enzymatic domain, the exogenous protease cleavage site, the targeting domain, the Clostridial toxin translocation domain, 3) the targeting domain, the Clostridial toxin translocation domain, the exogenous protease cleavage site and the Clostridial toxin enzymatic domain, 4) the targeting domain, the Clostridial toxin enzymatic domain, the exogenous protease cleavage site, the Clostridial toxin translocation domain, 5) the Clostridial toxin translocation domain, the exogenous protease cleavage site, the Clostridial toxin enzymatic domain and the targeting domain, or 6) the Clostridial toxin translocation domain, the exogenous protease cleavage site, the targeting domain and the Clostridial toxin enzymatic domain.

9. The method of claim 7, wherein the Clostridial toxin translocation domain is a BoNT/A translocation domain, a BoNT/B translocation domain, a BoNT/C1 translocation domain, a BoNT/D translocation domain, a BoNT/E translocation domain, a BoNT/F translocation domain, a BoNT/G translocation domain, a TeNT translocation domain, a BaNT translocation domain, or a BuNT translocation domain.

10. The method of claim 7, wherein the Clostridial toxin enzymatic domain is a BoNT/A enzymatic domain, a BoNT/B enzymatic domain, a BoNT/C1 enzymatic domain, a BoNT/D enzymatic domain, a BoNT/E enzymatic domain, a BoNT/F enzymatic domain, a BoNT/G enzymatic domain, a TeNT enzymatic domain, a BaNT enzymatic domain, or a BuNT enzymatic domain.

11. The method of claim 7, wherein the exogenous protease cleavage site is a plant papain cleavage site, an insect papain cleavage site, a crustacian papain cleavage site, an enterokinase cleavage site, a human rhinovirus 3C protease cleavage site, a human enterovirus 3C protease cleavage site, a tobacco etch virus protease cleavage site, a Tobacco Vein Mottling Virus cleavage site, a subtilisin cleavage site, a hydroxylamine cleavage site, or a Caspase 3 cleavage site.

12. The method of claim 7, wherein the sexual dysfunction disorder is a sexual desire disorder, a sexual arousal disorder, a sexual orgasm disorder, a sexual pain disorder, a sexsomnia, or a climacturia.

13. The method of claim 12, wherein the sexual desire disorder is a hypoactive sexual desire disorder, a sexual aversion disorder, or a hyperactive sexual desire disorder.

14. The method of claim 12, wherein the sexual arousal disorder is a deficient sexual arousal disorder, a persistent sexual arousal disorder, or a priapism.

15. The method of claim 12, wherein the sexual orgasm disorder is a male orgasmic disorder, a male anorgasmia, a premature ejaculation, an ejaculatory incompetence, a female orgasmic disorder, a female anorgasmia, or an inhibited female orgasm.

16. The method of claim 12, wherein the sexual pain disorder is a dyspareunia, a vaginismus, a vulvodynia, a dysorgasmia, or a testicular pain.

17. A use of a TEM in the manufacturing a medicament for treating a sexual dysfunction disorder in an individual in need thereof, wherein the TEM comprising a targeting domain, a Clostridial toxin translocation domain and a Clostridial toxin enzymatic domain, wherein the targeting domain is a sensory neuron targeting domain, a sympathetic neuron targeting domain, or a parasympathetic neuron targeting domain.

18. A use of a TEM in the manufacturing a medicament for treating a sexual dysfunction disorder in an individual in need thereof, wherein the TEM comprising a targeting domain, a Clostridial toxin translocation domain, a Clostridial toxin enzymatic domain, and an exogenous protease cleavage site, wherein the targeting domain is a sensory neuron targeting domain, a sympathetic neuron targeting domain, or a parasympathetic neuron targeting domain.
